(19) United States
(12) Patent Application Publication
(10) Pub. No.: US 2005/0287202 A1
(43) Pub. Date: Dec. 29, 2005

(54) Compound

(76) Inventors: Andrew David Miller, Chiswick (GB); Michael Keller, Villas (GB); Michael Jorgensen, London (GB); Eric Perouzel, London (GB)

Correspondence Address: Scott A. McCollister, Esq.;Fay, Sharpe, Fagan, Minnich & McKee, LLP, Seventh Floor, 1100 Superior Avenue, Cleveland, OH, 44114-2579, US

(21) Appl. No.: 11/159,938

(22) Filed: Jun. 23, 2005

Related U.S. Application Data
(63) Continuation-in-part of application No. 10/008,129, filed on Nov. 5, 2001.

(30) Foreign Application Priority Data
Dec. 12, 2000 (WO) PCT/GB00/04767

(51) Int. Cl. A61K 48/00; A61K 9/127

(52) U.S. Cl. 424/450; 514/44; 554/53

(57) ABSTRACT

The present invention provides a process for preparing a modified lipid of the formula 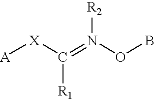 comprising reacting
(I) a compound of the formula; and 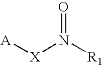
(ii) a compound of the formula 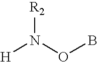 wherein component (ii) is formulated as a liposome; wherein B is a lipid; wherein A is a moiety of interest (MOI) and is a hydrocarbyl group; wherein X is an optional linker group;
wherein R₁ is H or a hydrocarbyl group; and
wherein R₂ is a lone pair, H or a hydrocarbyl group. The moiety of interest A may be selected from a carbohydrate moiety, a polymer, a peptide, a glycoprotein, a small biomolecule (such as a folic acid derivative) and a bioconjugate linker.

This application is a continuation-in-part filing claiming the priority benefit of U.S. application Ser. No. 10/008,129, filed Dec. 5, 2001, the disclosure of which is incorporated herein by reference.

The present invention relates to a compound.

One aspect of gene therapy involves the introduction of foreign nucleic acid (such as DNA) into cells, so that its expressed protein may carry out a desired therapeutic function.

Examples of this type of therapy include the insertion of TK, TSG or ILG genes to treat cancer; the insertion of the CFTR gene to treat cystic fibrosis; the insertion of NGF, TH or LDL genes to treat neurodegenerative and cardiovascular disorders; the insertion of the IL-1 antagonist gene to treat rheumatoid arthritis; the insertion of HIV antigens and the TK gene to treat AIDS and CMV infections; the insertion of antigens and cytokines to act as vaccines; and the insertion of β-globin to treat haemoglobinopathic conditions, such as thalassaemias.

Many current gene therapy studies utilise adenoviral gene vectors - such as Ad3 or Ad5 - or other gene vectors. However, serious problems have been associated with their use. This has prompted the development of less hazardous, non-viral approaches to gene transfer.

A non-viral transfer system of great potential involves the use of cationic liposomes. In this regard, cationic liposomes—which usually consist of a neutral phospholipid and a cationic lipid—have been used to transfer DNA, mRNA, antisense oligonucleotides, proteins, and drugs into cells. A number of cationic liposomes are commercially available and many new cationic lipids have recently been synthesised. The efficacy of these liposomes has been illustrated by both in vitro and in vivo.

A cytofectin useful in the preparation of a cationic liposome is N-[1-(2,3-dioleoyloxy)propyl]-N,N,N-trimethyl ammonium chloride, otherwise known as “DOTMA”.

One of the most commonly used cationic liposome systems consists of a mixture of a neutral phospholipid dioleoylphosphatidylethanolamine (commonly known as “DOPE”) and a cationic lipid, 3p-[(N,N-dimethylaminoethane)carbamoyl]cholesterol (commonly known as “DC-Chol”).

Despite the efficacy of the known cationic liposomes there is still a need to optimise the gene transfer efficiency of cationic liposomes in human gene therapy. With the near completion of the human genome project, the use of genes for therapeutic purposes, described as gene therapy is increasingly expected to revolutionise medicine. In this context, even though still less effective than viral technology, non-viral delivery is increasingly recognised by the scientific community as the safest option for human applications.

This field has evolved considerably in the last decade with the apparition of complex macromolecular constructs including many elements of different existing technologies (viral proteins or peptides, liposomes, polymers, targeting strategies and stealth properties).

Our copending application PCT/GB00/04767 teaches a system based on a triplex composed of a viral core peptide Mu, plasmid DNA and cationic Liposome (LMD). This platform technology gave us good success in vitro and promising results in vivo. But as for all existing non-viral technology more development is needed to achieve a therapeutic level in vivo.

To this end, formulation must achieve stability of the particle in biological fluids (serum, lung mucus) and still maintain efficient transfection abilities.

This requirement is one of the main hurdles of all existing technology. Current stable formulations achieve little transfection and most present efficient transfecting agents are drastically limited in the scope of their application due to this instability.

After administration (in blood for systemic application or in mucus for lung topical administration), the charged complexes are exposed to salt and biological macromolecules leading to strong colloidal aggregation and adsorption of biological active elements (opsonins) at their surface. The gene vehicles undergo drastic changes that could include precipitation, binding of proteins leading to particle elimination by macrophages and surface perturbation resulting in its destruction.

With the aim of generating drug and gene delivery systems for cell specific targeting in vitro and in vivo, protocols are required for the production of biological fluid-stable delivery systems with sufficient activity to exhibit therapeutic benefits. Therefore, a balance between stability and activity must be found for an efficient drug/gene delivery vehicle.

Our copending applications PCT/GB00/04767 teaches a system based on modified lipid wherein the lipid carries a carbohydrate moiety. These modified lipids have been found to stable and have low toxicity. Such systems require the linking an additional moiety to the lipid to assist in the provision of a modified lipid which is stable and has low toxicity. There is a desire in the art to provide lipids comprising groups to which additional moieties may be readily linked.

The present invention alleviates the problems of the prior art.

According to one aspect of the present invention there is provided a compound of the formula 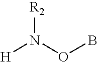
wherein B is a lipid; and wherein R₂ is H or a hydrocarbyl group.

According to one aspect of the present invention there is provided a process for preparing a modified lipid of the formula 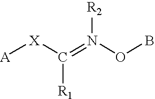
comprising reacting (i) a compound of the formula; and 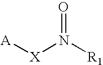
(ii) a compound of the formula 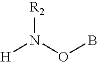
   wherein B is a lipid and A is a moiety of interest (MOI); wherein X is an optional linker group; wherein R₁ is H or a hydrocarbyl group; and wherein R₂ is a lone pair or R₄, wherein R₄ is a suitable substituent.

According to one aspect of the present invention there is provided a process for preparing a modified lipid of the formula 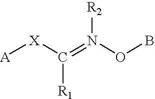
comprising reacting
(i) a compound of the formula; and 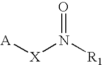
(ii) a compound of the formula 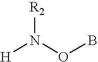
   wherein component (ii) is formulated as a liposome or as a component of a liposome;
   wherein B is a lipid;
   wherein A is a moiety of interest (MOI) and is a hydrocarbyl group;
   wherein X is an optional linker group;
   wherein R₁ is H or a hydrocarbyl group; and
   wherein R₂ is a lone pair, H or a hydrocarbyl group.

According to one aspect of the present invention there is provided a process for preparing a compound of the formula 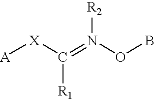
comprising reacting
(i) a compound of the formula; and 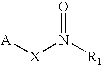
(ii) a compound of the formula 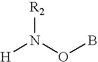
   in admixture with or associated with a nucleotide sequence, or a pharmaceutically active agent;
   wherein B is a lipid;
   wherein A is a moiety of interest (MOI) and is a hydrocarbyl group;
   wherein X is an optional linker group;
   wherein R₁ is H or a hydrocarbyl group; and
   wherein R₂ is a lone pair, H or a hydrocarbyl group.

According to one aspect of the present invention there is provided a composition comprising (i) a compound of the formula 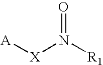
(ii) a compound of the formula 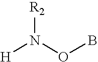
   wherein B is a lipid and A is a moiety of interest (MOI); wherein X is an optional linker group; wherein R₁ is H or a hydrocarbyl group; and wherein R₂ is a lone pair or a suitable substituent.

According to one aspect of the present invention there is provided a composition comprising
(i) a compound of the formula 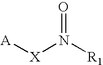
(ii) a compound of the formula 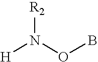
   wherein component (ii) is formulated as a liposome or as a component of a liposome;
   wherein B is a lipid;
   wherein A is a moiety of interest (MOI) and is a hydrocarbyl group;
   wherein X is an optional linker group;
   wherein R₁ is H or a hydrocarbyl group; and
   wherein R₂ is a lone pair, H or a hydrocarbyl group.

According to one aspect of the present invention there is provided a composition comprising
(i) a compound of the formula 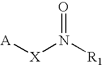
(ii) a compound of the formula 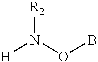
   and
(iii) a nucleotide sequence, or a pharmaceutically active agent;
   wherein B is a lipid;
   wherein A is a moiety of interest (MOI) and is a hydrocarbyl group;
   wherein X is an optional linker group;
   wherein R₁ is H or a hydrocarbyl group; and
   wherein R₂ is a lone pair, H or a hydrocarbyl group.

According to another aspect of the present invention there is provided a compound, a composition or a compound when prepared by the process of the present invention for use in therapy.

According to another aspect of the present invention there is provided the use of a compound, a composition or a compound when prepared by the process of the present invention in the manufacture of a medicament for the treatment of a genetic disorder or a condition or a disease.

According to another aspect of the present invention there is provided a liposome formed from a compound, a composition or a compound when prepared by the process of the present invention.

According to another aspect of the present invention there is provided a method of preparing a liposome comprising forming the liposome from a compound, a composition or a compound when prepared by the process of the present invention.

According to another aspect of the present invention there is provided a liposome according to the present invention or a liposome as prepared by the method of the present invention for use in therapy.

According to another aspect of the present invention there is provided the use of a liposome according to the present invention or a liposome as prepared by the method of the present invention in the manufacture of a medicament for the treatment of genetic disorder or condition or disease.

According to another aspect of the present invention there is provided a combination of a nucleotide sequence or a pharmaceutically active agent and any one or more of: a compound, a composition, a compound when prepared by the process of the present invention, a liposome of the present invention, or a liposome as prepared by the method of the present invention.

According to another aspect of the present invention there is provided a combination according to the present invention for use in therapy.

According to another aspect of the present invention there is provided the use of a combination according to the present invention in the manufacture of a medicament for the treatment of genetic disorder or condition or disease.

According to another aspect of the present invention there is provided a pharmaceutical composition comprising a compound, a composition or a compound when prepared by the process of the present invention admixed with a pharmaceutical and, optionally, admixed with a pharmaceutically acceptable diluent, carrier or excipient.

According to another aspect of the present invention there is provided a pharmaceutical composition comprising a liposome according to the present invention or a liposome as prepared by the method of the present invention admixed with a pharmaceutical and, optionally, admixed with a pharmaceutically acceptable diluent, carrier or excipient.

Some further aspects of the invention are defined in the appended claims.

We have found the provision of a lipid comprising an aminoxy group allows for simple linking of further moieties to the lipid via the aminoxy group. When reacted with a moiety (MOI) comprising an aldehyde or ketone group, a compound is provided in which the MOI and lipid are linked via an amide group. Such a linkage may be simple prepared in a “one-pot” reaction. This methodology avoids extensive purification procedures by simple dialysis of excess, non-reacted reagents.

The post-coating one-pot methodology of the present process is based on selective and high reactivity of the aminoxy-linker to react with aldehydes and ketones to form —C═N-(Schiff-base like) covalent linkages. Importantly, the reaction can be carried out in aqueous environment at basic or acidic pH. Furthermore, there is no partial breakdown of the reactive group when exposed to aqueous conditions as it is the case for NHS-activated carboxyls and other esters. Therefore, the stability of the reactive species, e.g. the aldehyde/ketone and the aminoxy allows total control of the surface reaction without loss of reactive species due to hydrolysis/degradation.

### PREFERRED ASPECTS

Component (ii) of the present invention is a compound of the formula 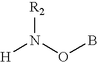
wherein B is a lipid; and wherein R₂ is H or a hydrocarbyl group.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

Preferably components (i) and (ii) are in admixture with or associated with a nucleotide sequence, or a pharmaceutically active agent.

Preferably component (ii) is formulated as a liposome or as a component of a liposome.

Preferably the reaction of the present invention is performed in an aqueous medium.

### Optional Linker X

In a preferred aspect optional linker X is present.

In a preferred aspect X is a hydrocarbyl group.

In a preferred aspect the linker X comprises or is linked to the lipid via a polyamine group.

It is believed that the polyamine group is advantageous because it increases the DNA binding ability and efficiency of gene transfer of the resultant liposome.

In one embodiment, preferably the polyamine group is a unnaturally occurring polyamine. It is believed that the polyamine head-group is advantageous because the increased amino functionality increases the overall positive charge of the liposome. In addition, polyamines are known to both strongly bind and stabilise DNA. In addition, polyamines occur naturally in cells and so it is believed that toxicological problems are minimised.

In another embodiment, preferably two or more of the amine groups of the polyamine group of the present invention are separated by one or more groups which are not found in nature that separate amine groups of naturally occurring polyamine compounds (i.e. preferably the polyamine group of the present invention has un-natural spacing).

Preferably the polyamine group contains at least two amines of the polyamine group that are separated (spaced from each other) from each other by an ethylene (—CH₂CH₂—) group.

Preferably each of the amines of the polyamine group are separated (spaced from each other) by an ethylene (—CH₂CH₂—) group.

Typical examples of suitable polyamines include spermidine, spermine, caldopentamine, norspermidine and norspermine. Preferably the polyamine is spermidine or spermine as these polyamines are known to interact with single or double stranded DNA. An alternative preferred polyamine is caldopentamine.

In a preferred embodiment, the linker X comprises a polyether group.

Preferably the polyether group comprises at least two oxygen atoms separated (spaced from each other) from each other by an alkyl group.

In one embodiment the polyether group comprises at least two oxygen atoms separated (spaced from each other) from each other by a first alkyl group, and at least two oxygen atoms separated (spaced from each other) from each other by a second alkyl group.

In another embodiment, each of the oxygen atoms of the polyether are separated from each other by alkyl groups of the same type.

Preferably the alkyl group is selected from methylene, ethylene, propylene and butylene.

Preferably the alkyl group is ethylene.

Typical examples of suitable polyethers are polyethylene glycol (PEG) polymers.

Preferably the polyether group comprises a number average molecular weight of from about 44 to about 10,000. Preferably the polyether group comprises a number average molecular weight of from about 1,000 to about 9,000; from about 1,500 to about 7,000; from about 2,000 to about 5,000.

R₁

In a preferred aspect R₁ is H

C═N

The C═N bond may be acid labile or acid resistant.

In one aspect the C═N bond is acid labile.

In one aspect the C═N bond is acid resistant.

A

A is a moiety of interest. The moiety of interest (MOI) may be any moiety which one wishes to link to a lipid. This may be any molecule of biological interest.

Preferably A is a hydrocarbyl group.

Preferably A is selected from a carbohydrate moiety, a polymer, a peptide, a glycoprotein, a small biomolecule and a bioconjugate linker.

The MOI may be a carbohydrate moiety.

In a preferred aspect the carbohydrate moiety is a mono-saccharide.

In a preferred aspect the carbohydrate moiety is a sugar moiety.

Preferably the carbohydrate moiety is selected from mannose, glucose (D-glucose), galactose, glucuronic acid, lactose, maltose, maltotriose, maltotetraose, maltoheptaose and mixtures thereof. More preferably the carbohydrate moiety is D-glucose.

In one aspect the compound of the present invention comprises from 1 to 7 carbohydrate moieties. Preferably the compound comprises one carbohydrate moiety.

Preferably A is a polymer. Preferably the polymer is a polyether polymer. Preferably the polyether group comprises a number average molecular weight of from about 44 to about 10,000. Preferably the polyether group comprises a number average molecular weight of from about 1,000 to about 9,000; from about 1,500 to about 7,000; from about 2,000 to about 5,000.

Preferably A is a polyethylene glycol. Preferably the polyethylene glycol is derived from PEG₂₀₀₀ bis-propionaldehyde.

Preferably the peptide comprises an RGD peptide. Preferably the RGb peptide is an agonist for αᵥβ₃ integrins. An RGD peptide is one comprising the amino acid sequence Arg-Gly-Asp. This sequence is present in extracellular matrix proteins such as fibronectin.

Preferably the peptide is transferrin.

Preferably the peptide is an antibody. Suitable antibodies may include those disclosed in U.S. Pat. No. 5,332,567.

Preferably A is a glycoprotein. Glycoproteins comprise a protein and a carbohydrate joined together in a covalent chemical linkage. Preferably the carbohydrate of the glycoprotein comprises glucose, glucosamine, galactose, galactosamine, mannose, fucose and sialic acid.

Preferably A is a small biomolecule. Preferably A is a small biomolecule selected from folic acid and a folic acid derviative. Preferably the folic acid derviative is an ester of folic acid or a pharmaceutically acceptable salt thereof.

Preferably A is a bioconjugate linker. Preferably A is a bioconjugate linker selected from an aldehyde, an amine, a thiocyanate, an isocyanate and a maleimide group.

B

B is a lipid.

Preferably B comprises a lipid of the formula: -W-Y-Z;

wherein W comprises a group selected from a polyamine group, a polyether group and mixtures thereof;

wherein Y is a linkage group; and

wherein Z is selected from a steroid, an acyl glcerol, a phosphoglceride, a ceramide and an acetamide derivative.

W

In one embodiment, preferably W comprises a polyamine group.

Preferably the polyamine group is a unnaturally occurring polyamine. Preferably two or more of the amine groups of the polyamine group of the present invention are separated by one or more groups which are not found in nature that separate amine groups of naturally occurring polyamine compounds (i.e. preferably the polyamine group of the present invention has un-natural spacing).

Preferably the polyamine group contains at least two amines of the polyamine group that are separated (spaced from each other) from each other by an ethylene (—CH₂CH₂—) group.

Preferably each of the amines of the polyamine group are separated (spaced from each other) by an ethylene (—CH₂CH₂—) group.

Preferably the polyamine group is selected from spermidine, spermine, caldopentamine, norspermidine and norspermine. Preferably the polyamine is spermidine or spermine as these polyamines are known to interact with single or double stranded DNA. An alternative preferred polyamine is caldopentamine.

In another embodiment, preferably W comprises a polyether group.

Preferably the polyether group comprises at least two oxygen atoms separated (spaced from each other) from each other by an alkyl group.

In one embodiment the polyether group comprises at least two oxygen atoms separated (spaced from each other) from each other by a first alkyl group, and at least two oxygen atoms separated (spaced from each other) from each other by a second alkyl group.

In another embodiment, each of the oxygen atoms of the polyether are separated from each other by alkyl groups of the same type.

Preferably the alkyl group is selected from methylene, ethylene, propylene and butylene.

Preferably the alkyl group is ethylene.

Typical examples of suitable polyethers are polyethylene glycol (PEG) polymers.

Y

Preferably the Y is linkage group selected from an ester, amide, carbamate and ether group.

Preferably Y is a carbamate group.

Z

Preferably Z is a steroid.

Preferably the steroid is selected from cholesterol, testosterone, androsterone, estrone, estradiol, progesterone, aldosterone, hydrocortisone, cortisone, bile acids and derivatives thereof.

Examples of steroid derivatives include substituted derivatives wherein one or more of the cyclic CH₂ or CH groups and/or one or more of the straight-chain CH₂ or CH groups is/are appropriately substituted. Alternatively, or in addition, one or more of the cyclic groups and/or one or more of the straight-chain groups may be unsaturated.

Preferably the steroid is cholesterol.

Preferably Z is an acyl glycerol. Preferably the acyl glycerol comprises at least one long chain hydrocarbyl group.

Preferably Z is a phosphoglyceride. Preferably the phosphoglyceride comprises at least one long chain hydrocarbyl group.

Preferably Z is a ceramide. Preferably the ceramide comprises at least one long chain hydrocarbyl group.

Preferably Z comprises an acetamide derivative. Preferably the acetamide derivative is a dialkyl substituted acetamide derivative of the formula —C(O)—NR₁₀OR₁₁, wherein R₁₀ and R₁₁ are independently selected from H and a long chain hydrocarbyl group.

### Lipid

In a preferred aspect the lipid is or comprises a cholesterol group or a glycerol/ceramide backbone. Any lipid-like structure or polyamine is suitable.

Preferably the cholesterol group is cholesterol.

Preferably the cholesterol group is linked to X via a carbamoyl linkage.

The cholesterol group can be cholesterol or a derivative thereof. Examples of cholesterol derivatives include substituted derivatives wherein one or more of the cyclic CH₂ or CH groups and/or one or more of the straight-chain CH₂ or CH groups is/are appropriately substituted. Alternatively, or in addition, one or more of the cyclic groups and/or one or more of the straight-chain groups may be unsaturated.

In a preferred embodiment the cholesterol group is cholesterol. It is believed that cholesterol is advantageous as it stabilises the resultant liposomal bilayer.

Preferably the cholesterol group is linked to the optional linker group via a carbamoyl linkage. It is believed that this linkage is advantageous as the resultant liposome has a low or minimal cytotoxicity.

### Long Chain Hydrocarbyl Group

Preferably the or each long chain hydrocarbyl group comprises between 5 and 50 carbon atoms; between 10 and 40 carbon atoms; between 15 and 35 carbon atoms.

Preferably the long chain hydrocarbyl group is a hydrocarbon group. Preferably the or each hydrocarbon group is independently selected from a satuarated, mono-unsaturated, or poly-unsaturated hydrocarbon group.

### Component (i)

In one preferred aspect, A is a bioconjugate linker, and linker X comprises a polyether group. Preferably A is a bioconjugate linker selected from an aldehyde, an amine, a thiocyanate, an isocyanate and a maleimide group. Preferably X is a polyether group as described hereinabove. More preferably, A is an aldehyde and X is a polyethylene glycol (PEG) polymer.

### Component (ii)

Preferably component (ii) is a compound of the formula

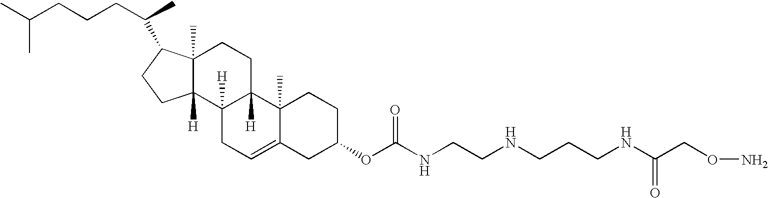

Preferably component (ii) is a compound of the formula

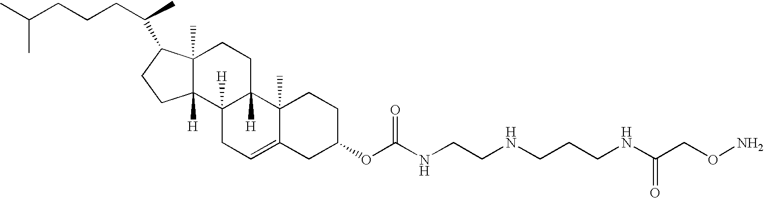

Preferably component (ii) is a compound of the formula

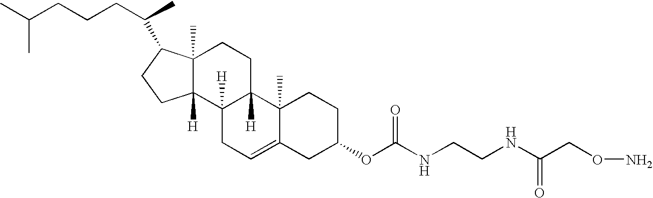

Preferably component (ii) is a compound of the formula

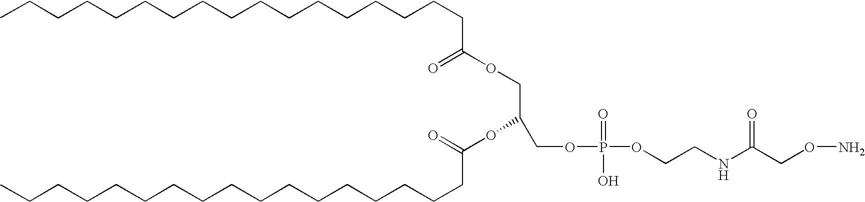

Preferably component (ii) is a compound of the formula

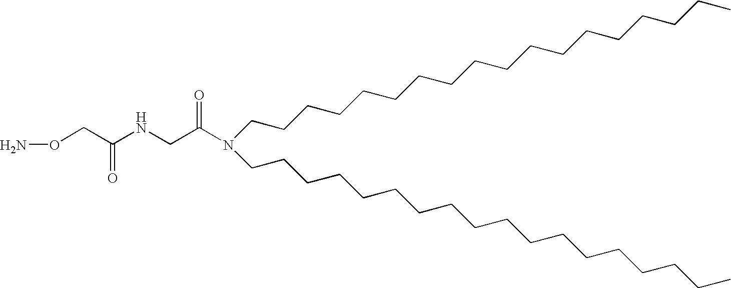

Preferably component (ii) is a compound of the formula 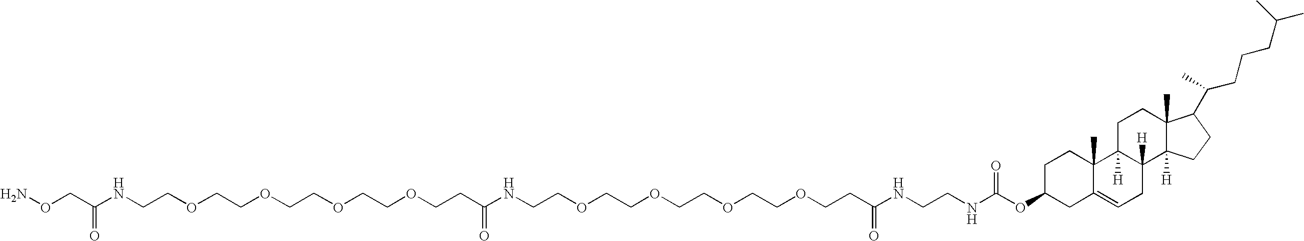
Further Aspects

Preferably R₂ is H or a hydrocarbyl group.

In a preferred aspect the R₂ hydrocarbyl group contains optional heteroatoms selected from O, N and halogens.

In a preferred aspect R₂ is H.

Preferably the process of the present invention is an aqueous medium or in a wholly aqueous medium.

The present invention further provide a compound prepared by a process of the present invention defined herein, a compound obtained by a process of the present invention defined herein, and/or a compound obtainable by a process of the present invention defined herein.

Preferably the compound is in admixture with or associated with a nucleotide sequence.

The nucleotide sequence may be part or all of an expression system that may be useful in therapy, such as gene therapy.

In a preferred aspect the compound of the present invention is in admixture with a condensed polypeptide/ nucleic acid complex to provide a non-viral nucleic acid delivery vector. The condensed polypeptide/ nucleic acid complex preferably include those disclosed in our copending application PCT/GB00/04767. Preferably the polypeptides or derivatives thereof are capable of binding to the nucleic acid complex. Preferably the polypeptides or derivatives thereof are capable of condensing the nucleic acid complex. Preferably the nucleic acid complex is heterologous to the polypeptides or derivatives thereof.

Preferably the process comprises the use of a molecular sieve.

Preferably, the cationic liposome is formed from the compound of the present invention and a neutral phospholipid—such as DOTMA or DOPE. Preferably, the neutral phospholipid is DOPE.

The present invention will now be described in further detail by way of example only with reference to the accompanying figures in which:

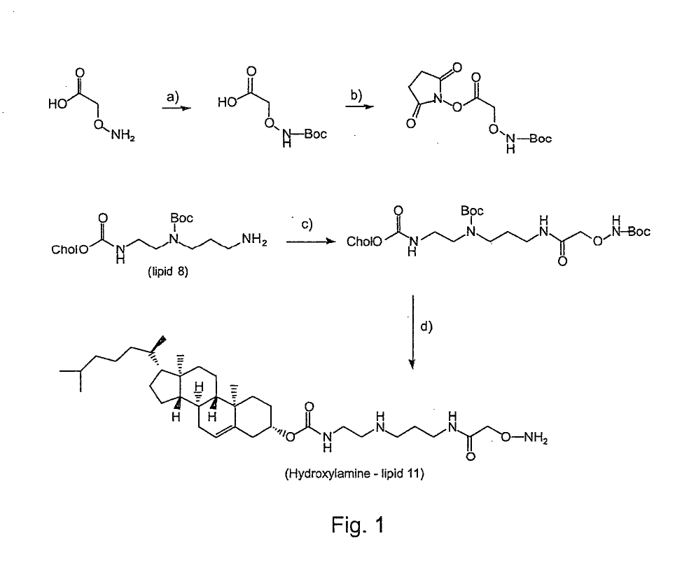

FIG. 1—Scheme 1 Synthesis of Hydroxylamine lipid 11. Reagents: (a) CH₂Cl₂, Et₃N, Boc₂O, rt, 5h, 98%; (b) EtOAc, N-hydroxysuccinimide (1 eq.), DCC (1 eq.), 10 h., rt; (c) (8), EtOAC/THF [95/5], Et₃N (pH=8), 2 h., r.t, 90%; (d) CH₂Cl₂, TFA (15 eq), 0° C., N₂, 5 h, 86%.

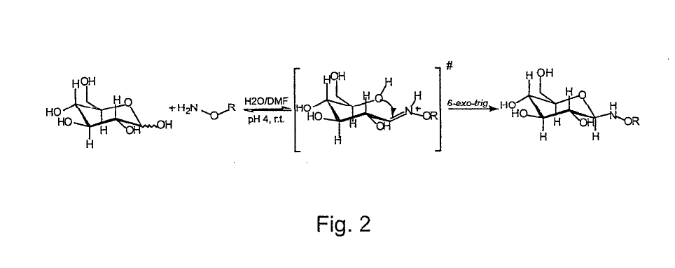

FIG. 2—Principle of chemioselective glycosylation of O-substituted hydroxylamine with D-Glucose (Although the P-anomer is shown, mutarotation does occur and α-anomer is produced as well).

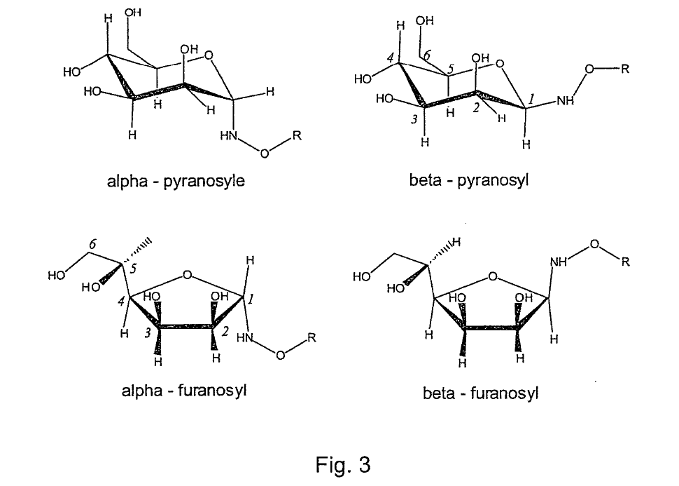

FIG. 3—Possible structures of neoglycolipid obtained from mannose.

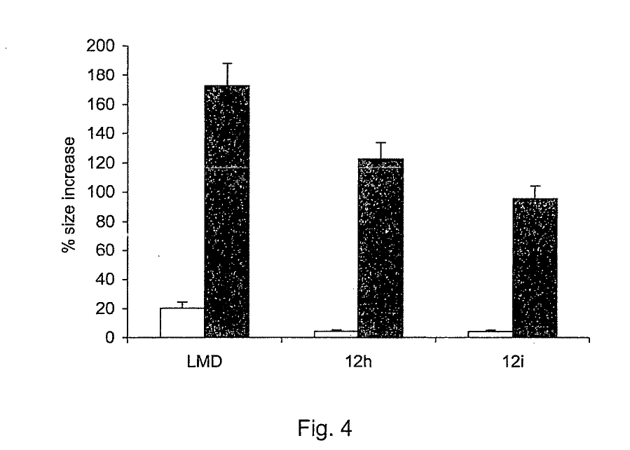

FIG. 4—Result of analysis of differents lipoplexes size by photon correlation spectroscopy (PCS). The size was measured after 30 min for lipoplexes at [DNA]=1 μg/ml in Optimem+/−10% FCS, 37° C. The comparison of standard LMD formulation (LMD) and LMD modified by addition of 7.5 molar % of product 12h and 12i was made in Optimem (white) and 10% Serum (black) and expressed in percent of size increase over the original measured size of 180 nm.

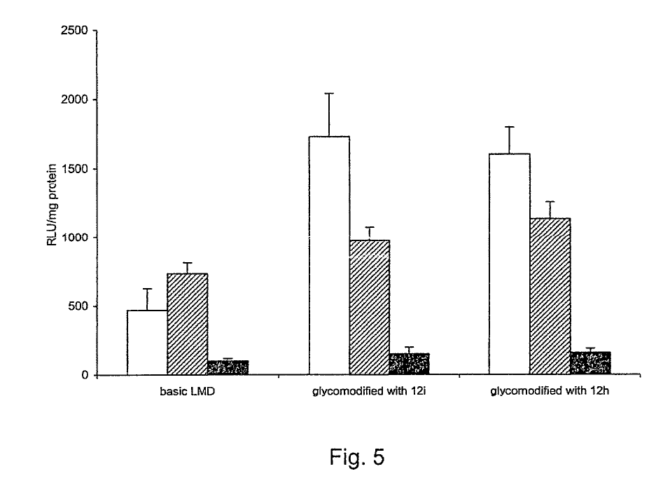

FIG. 5—A comparison between the transfection efficiencies of basic LMD and LMD glycomodified with 7.5 molar % of product 12h and 12i onto Hela Cells in 0% (white), 50% (black and white) and 100% Serum (black) conditions. The results are expressed as relative light units per milligram of protein (n=4).

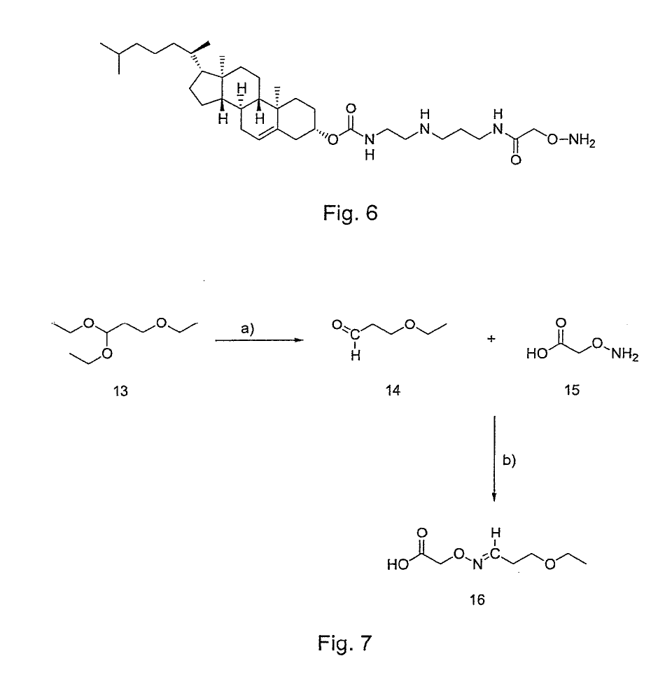

FIG. 6—A structure of an aminoxy lipid

FIG. 7—Synthesis of oxime 16. Reagents: (a) 1M HCl, THF, 0° C. to rt, 1 h.; (b) 15 (1 eq), CDCl₃/DMSO (3:1).

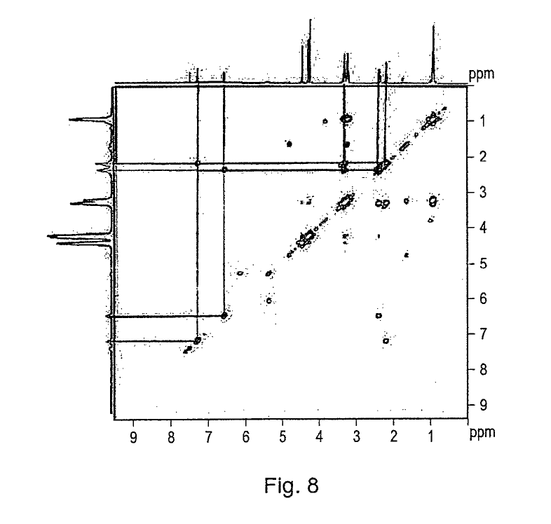

FIG. 8—¹H—¹H COSY NMR of oxime 16.

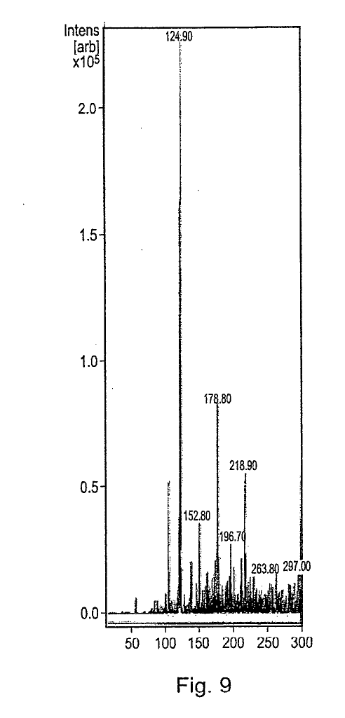

FIG. 9—Mass Spectrum of aldehyde 14.

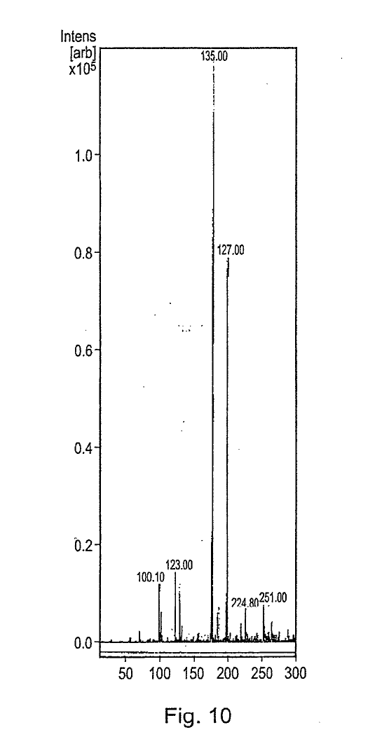

FIG. 10—Mass Spectrum of oxime 16.

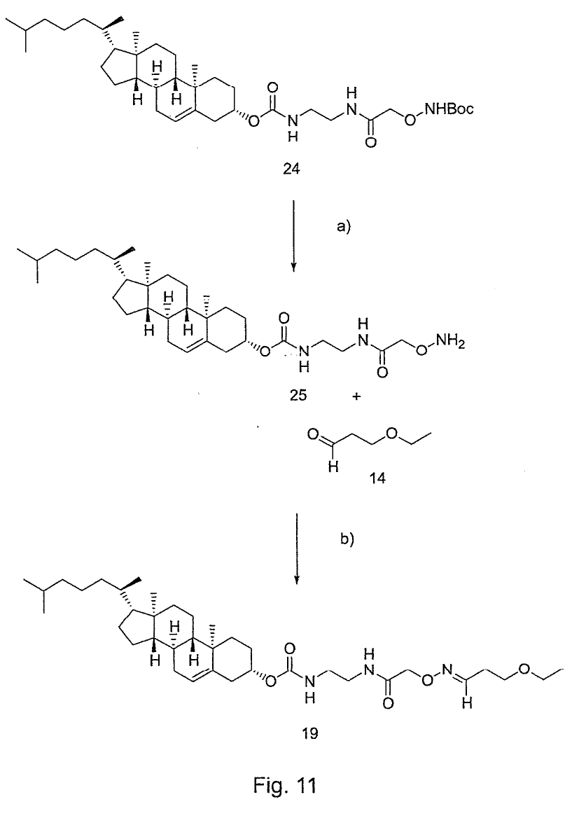

FIG. 11—Synthesis of oxime 19. Reagents: (a) 4M HCl, dioxane, propan-2-ol, rt, 3 h.; (b) 14 (1 eq), CDCl₃.

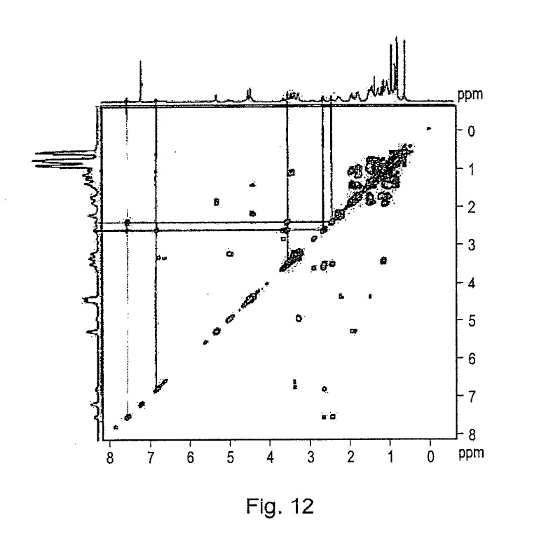

FIG. 12—¹H—¹H COSY NMR of oxime 19.

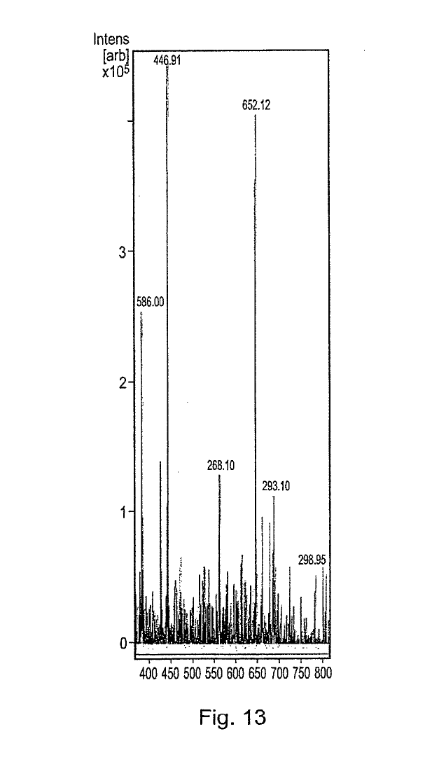

FIG. 13—Mass Spectrum of oxime 19.

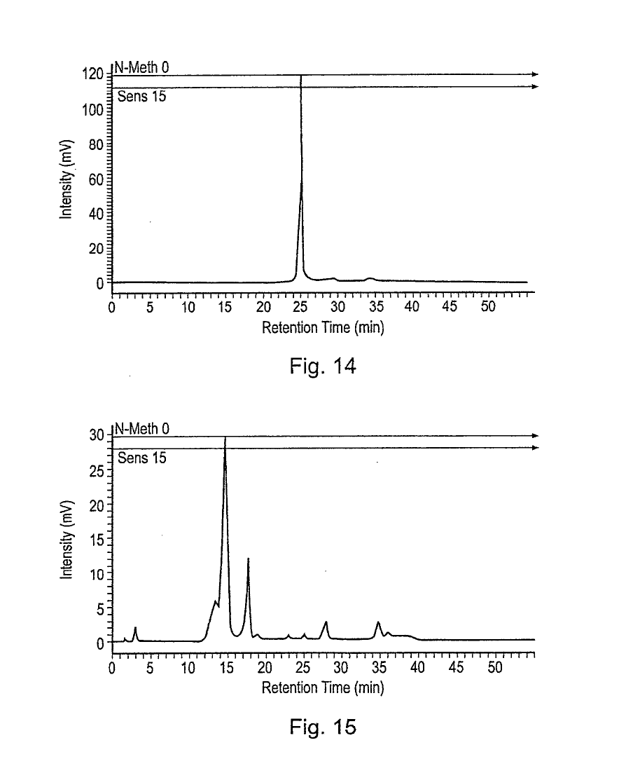

FIG. 14—HPLC of aminoxy-lipid 18.

FIG. 15—HPLC of aldehyde 14.

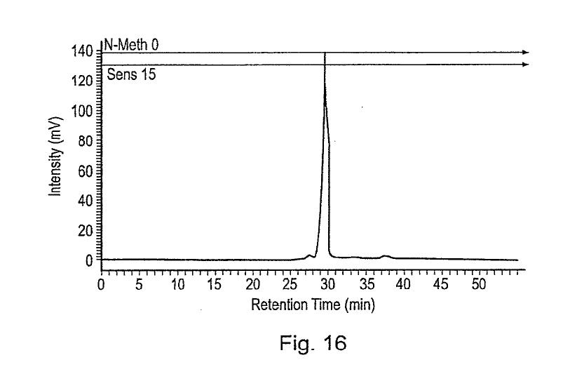

FIG. 16—HPLC of oxime 19.

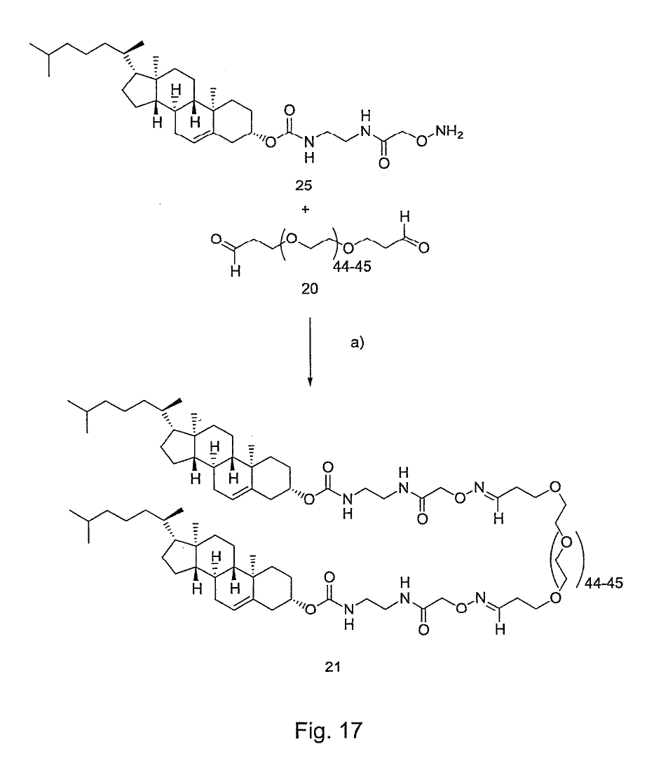

FIG. 17—Synthesis of oxime 21. Reagents: (a) 25 (2 eq), 20 (1 eq), CDCl₃, 12 h.

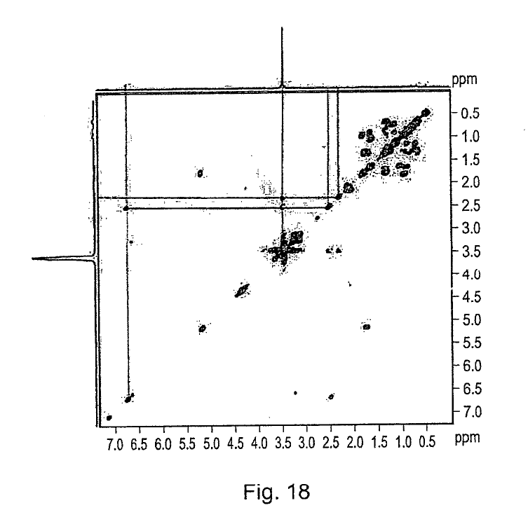

FIG. 18—¹H-¹H COSY NMR of oxime 21 from the solution reaction.

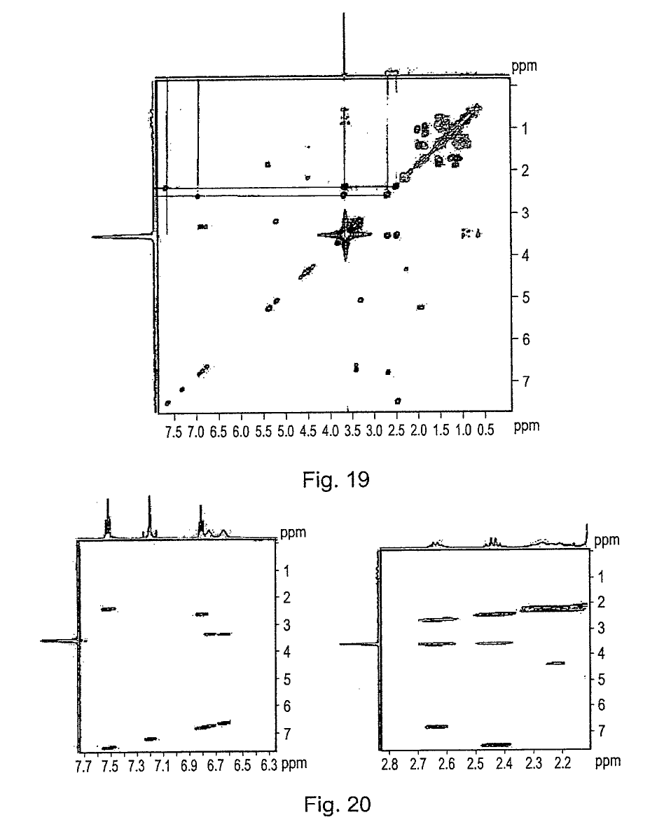

FIGS. 19 & 20—¹H-¹H COSY NMR of oxime 21 from the post-coupling liposome reaction.

FIG. 21—HPLC of for PEG₂₀₀₀-bis-propionaldehyde™ 20.

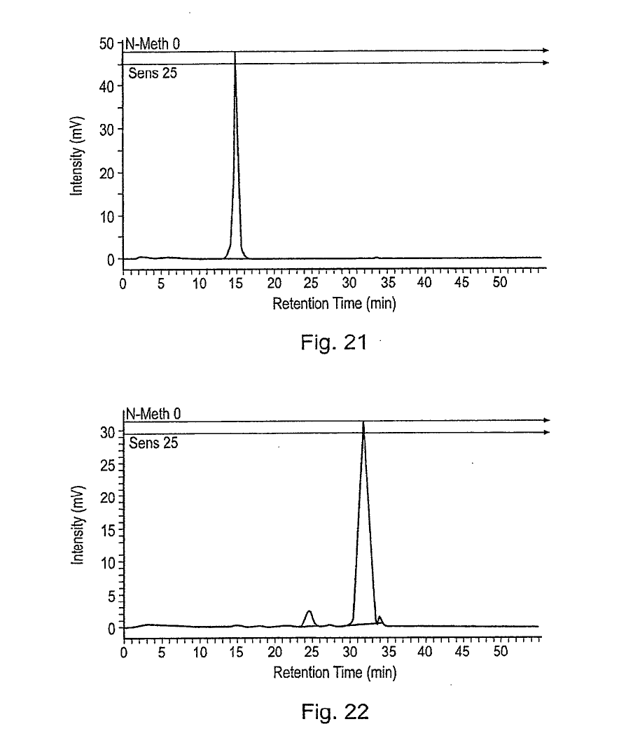

FIG. 22—HPLC of oxime 21.

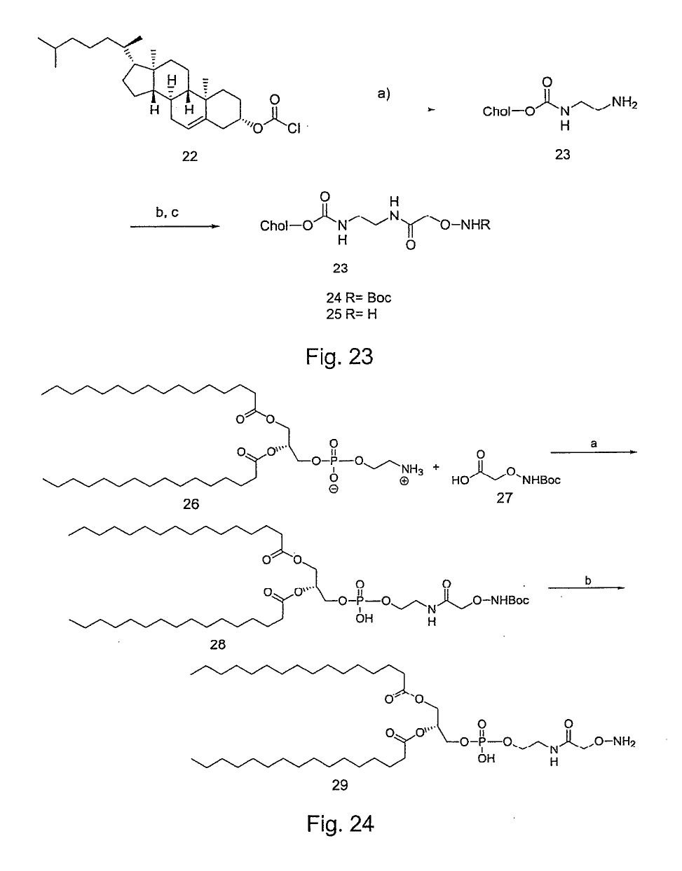

FIG. 23—Synthesis of Cholesteryl-aminoxy lipid 25. Reagents: a) ethylene diamine (large excess), r.t., 18 h, 78%; b) Boc-amino-oxyacetic acid, HBTU, DMAP, methylene chloride, r.t., 18 h, 81% and c) 4M HCl/dioxane, propan-2-ol, 3 h, 99%.

FIG. 24—Synthesis of DSPE-aminoxy lipid 29. Reagents: a) Boc-amino-oxyacetic acid, HBTU, DMAP, methylene chloride, r.t., 15 h, 56% and c) 4M HCI/ dioxane, propan-2-ol, 3 h, 41%.

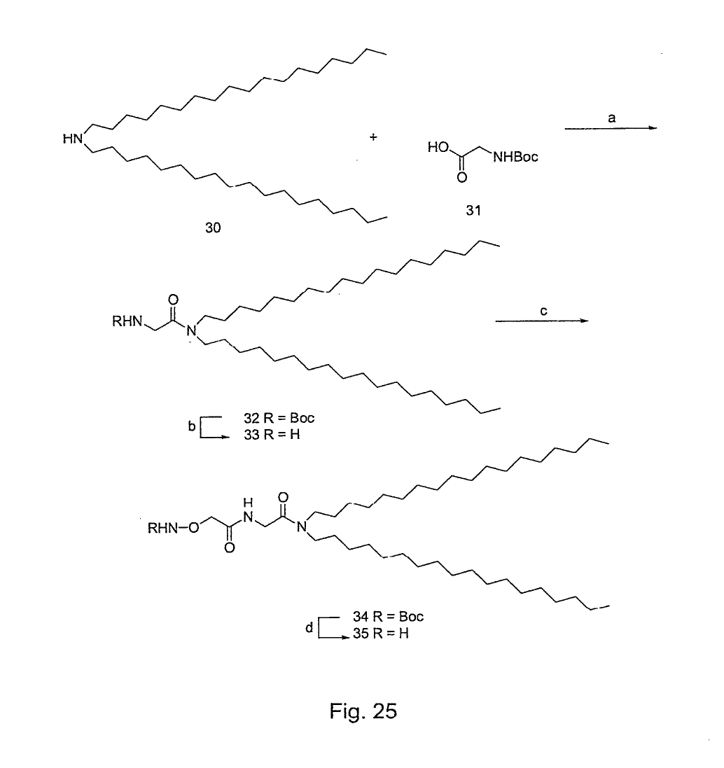

FIG. 25—Synthesis of 2-Aminooxy-N-dioctadecylcarbamoylmethyl-acetamide 35. Reagents: a) HBTU, DMAP, methylene chloride, r.t., 12 h, 70%; b) TFA:DCM (1:1 v/v), 2 h., r.t., 92%; c) Boc-amino-oxyacetic acid 27, HBTU, DMAP, methylene chloride, r.t., 14 h, 85% and d) TFA:DCM (1:1 v/v), 15 min., r.t., 100%.

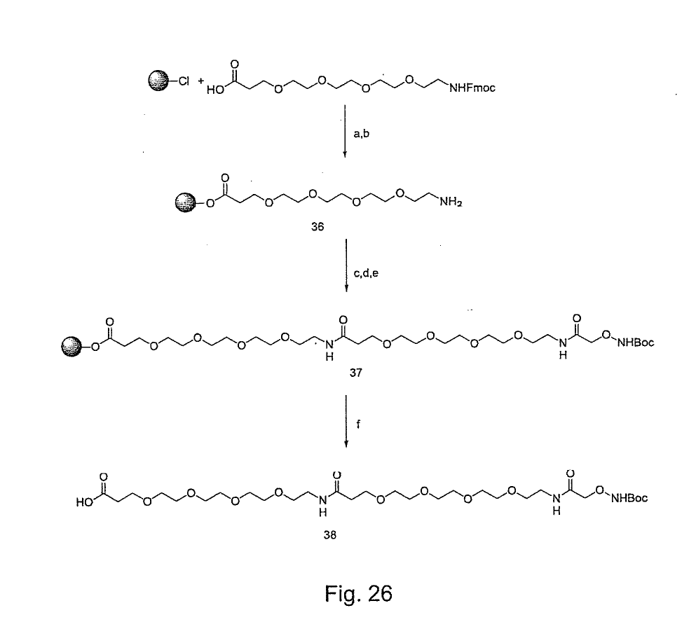

FIG. 26—Synthesis of Boc-aminoxy-(dPEG₄)₂-CO₂H 38. Reagents: a) N-Fmoc-amido-dPEG₄™-acid (3 equiv.), Hunig base (5 equiv.) in DMF, 2 h., r.t.; b) 20% Piperidine in DMF (3×5 min), r.t.; c) N-Fmoc-amido-dPEG₄™-acid (3 equiv.), HBTU (5 equiv.), Hunig base (5 equiv.) in DMF, 1 h., r.t.; d) 20% Piperidine in DMF (3×5 min), r.t.; e) Boc-amino-oxyacetic acid (3 equiv.), HBTU (5 equiv.), Hunig base (5 equiv.) in DMF, 1 h., r.t.; and f) 50% 1,1,1-trifluoroethanol in DCM, 1 h, r.t.

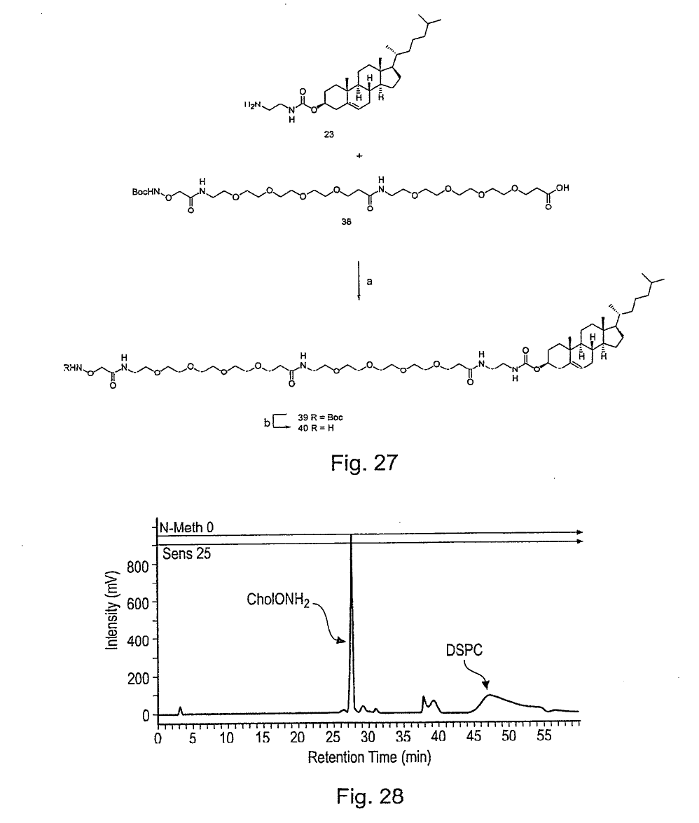

FIG. 27—Synthesis of CPA lipid 40. Reagents: a) Boc-amino-oxyacetic acid 27, HBTU, DMAP, methylene chloride, r.t., 18 h, 81% and b) 4M HCl/dioxane, propan-2-ol, 3 h, 99%.

FIG. 28—HPLC of liposome DSPC: CholONH₂ 25 (50:50).

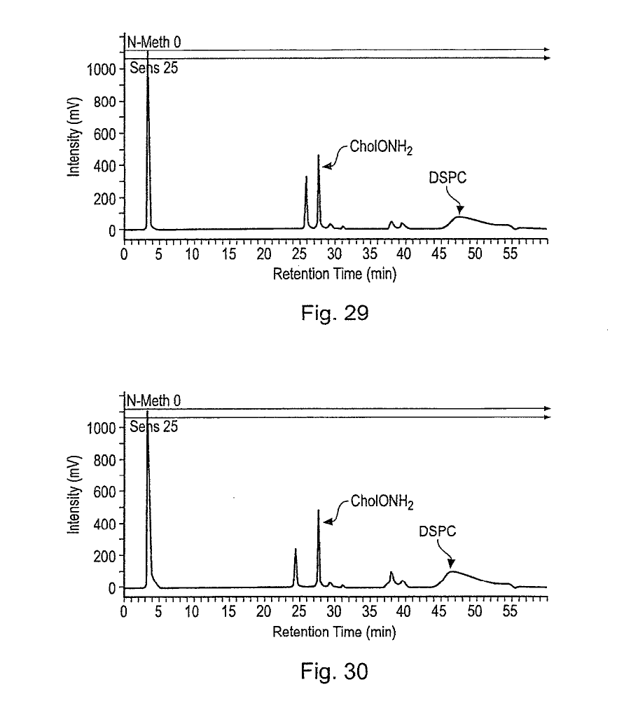

FIG. 29—HPLC of the reaction of liposome DSPC: CholONH₂ 25 (50:50) with lactose.

FIG. 30—HPLC of the reaction of liposome DSPC: CholONH₂ 25 (50:50) with maltoheptaose.

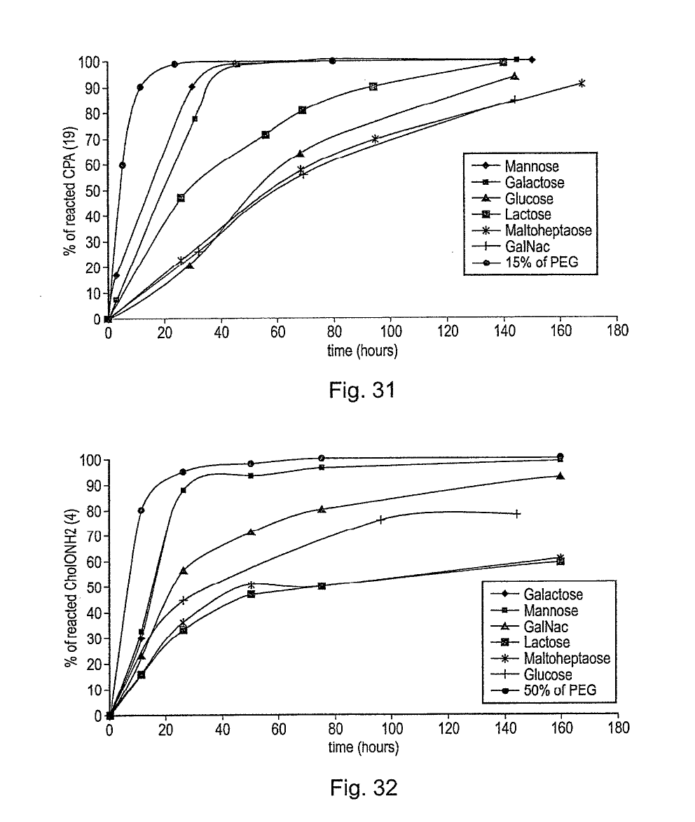

FIG. 31—Graph of the kinetics of coupling of liposome DSPC:CHOL:CPA 40 onto reduced carbohydrates or PEG²⁰⁰⁰(CHO)₂ at pH 5, 37° C.

FIG. 32—Graph of the kinetics of coupling of liposome DSPC:CholONH₂ 25 onto reduced carbohydrates or PEG²⁰⁰⁰(CHO)₂ at pH 5, 37° C.

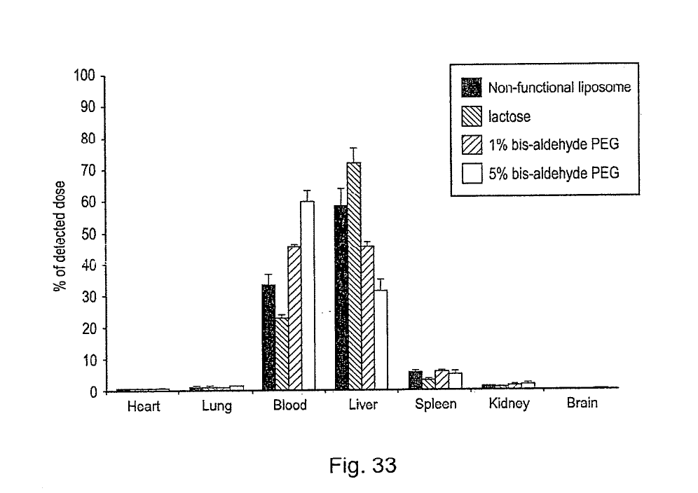

FIG. 33—Organ distribution of post-coupling liposomes incorporating aminoxy-lipid CPA 40 modified with PEG²⁰⁰⁰(CHO)₂ or lactose 30 mins after injection into mice.

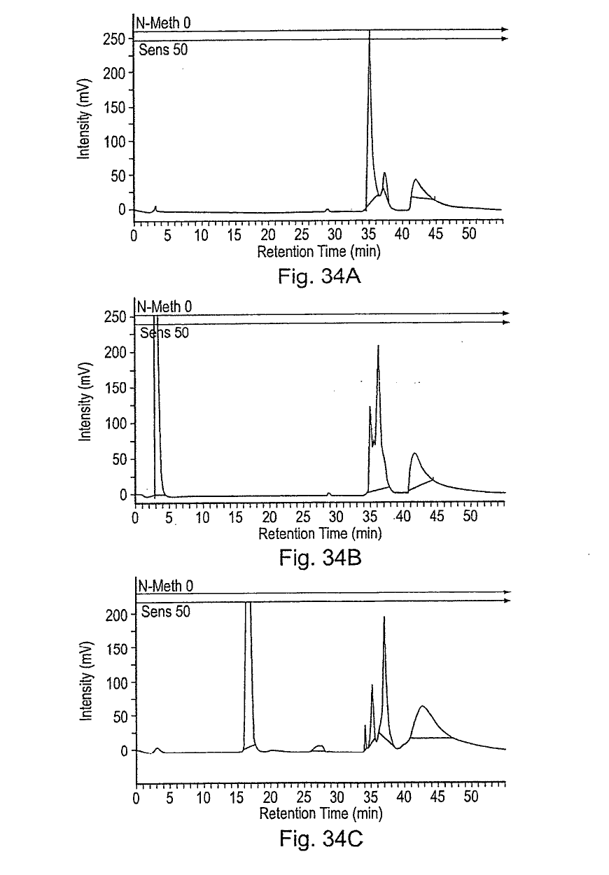

FIG. 34—A—HPLC of lipid 35: DSPC liposome; B—HPLC reaction of this liposome with galactose; C—HPLC reaction of this liposome with PEG.

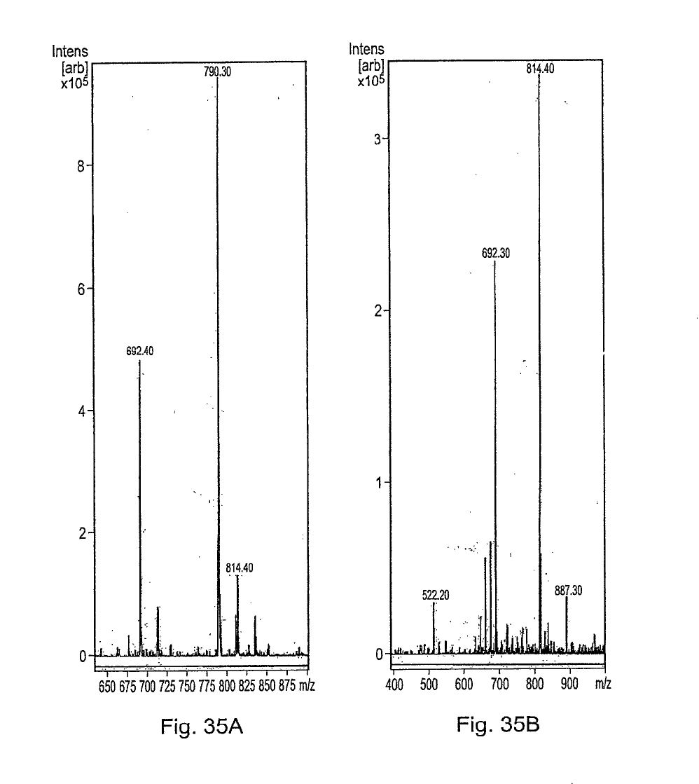

FIG. 35—A—Mass Spectrum of reaction of lipid 35:liposome with galactose; B—Mass Spectrum of coupled product of the reaction isolated by HPLC.

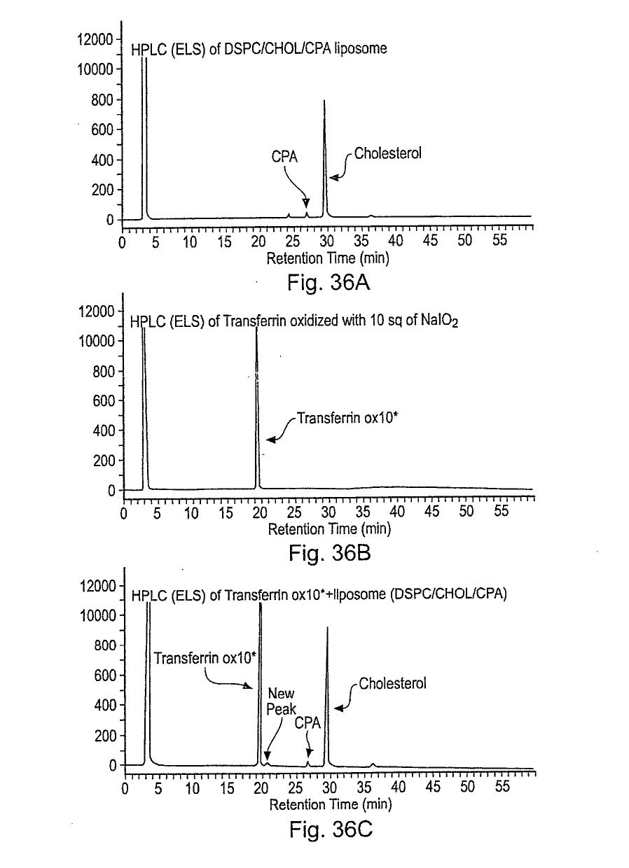

FIG. 36—A—HPLC of DSPC/CHOUCPA liposome 40; B—HPLC of transferrin oxidised with 10 eq of NalO₄; C—HPLC of reaction of the liposome with the oxidised transferrin.

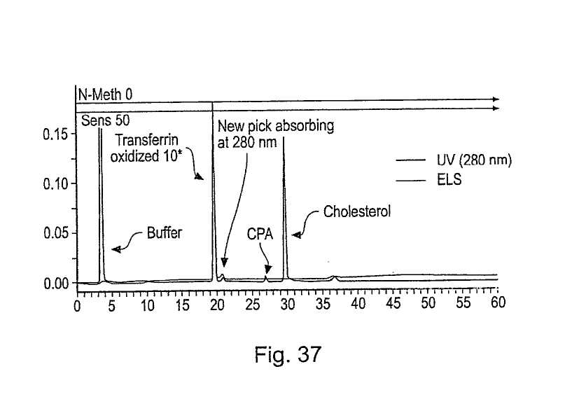

FIG. 37—HPLC of reaction of 40 with oxidised transferrin overlayed with UV (280 nm).

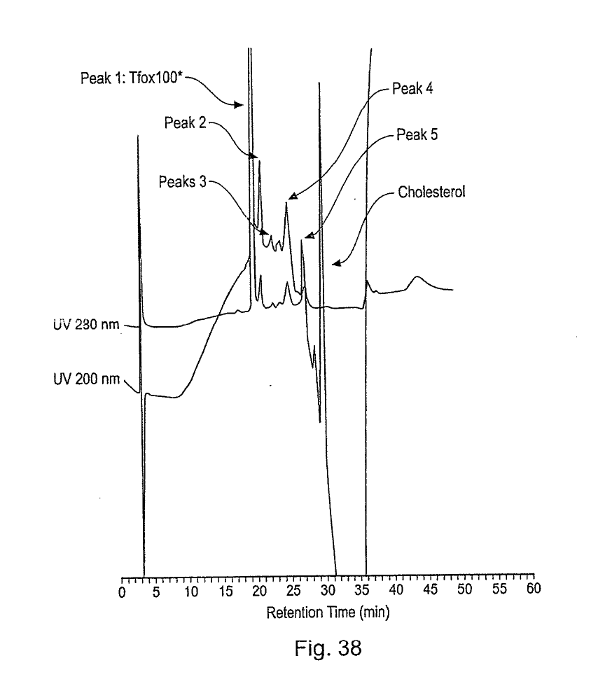

FIG. 38—HPLC of reaction of 40 with of transferrin oxidised with 100 eq of NalO₄.

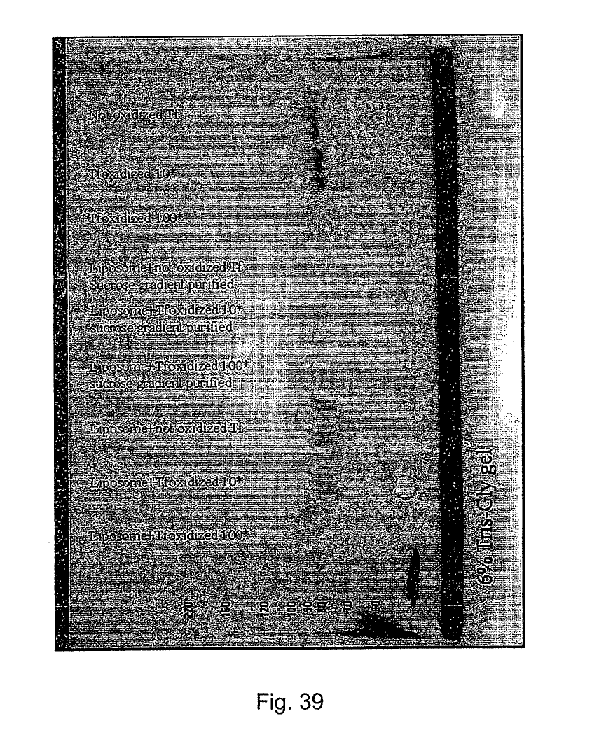

FIG. 39—Protein gel of HPLC isolated fraction of liposome 40 coupled to oxidised transferrin.

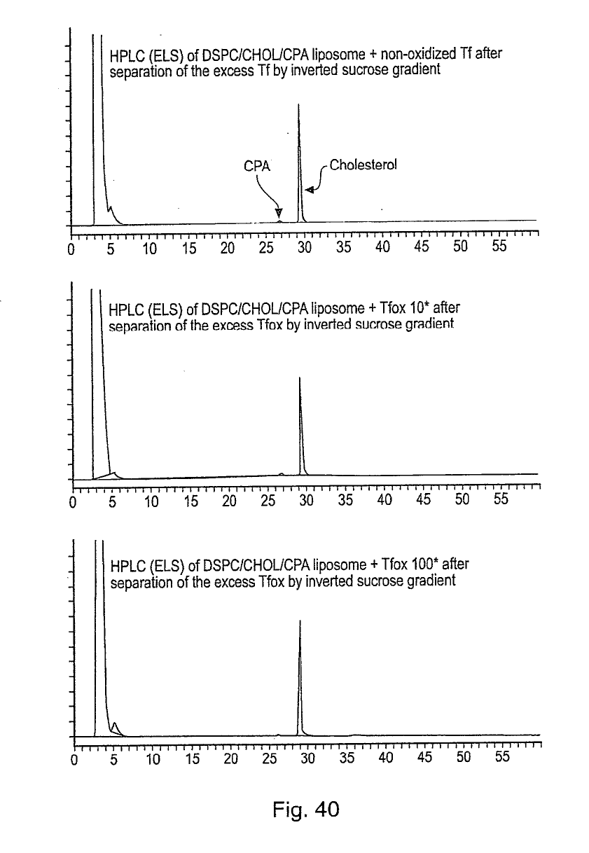

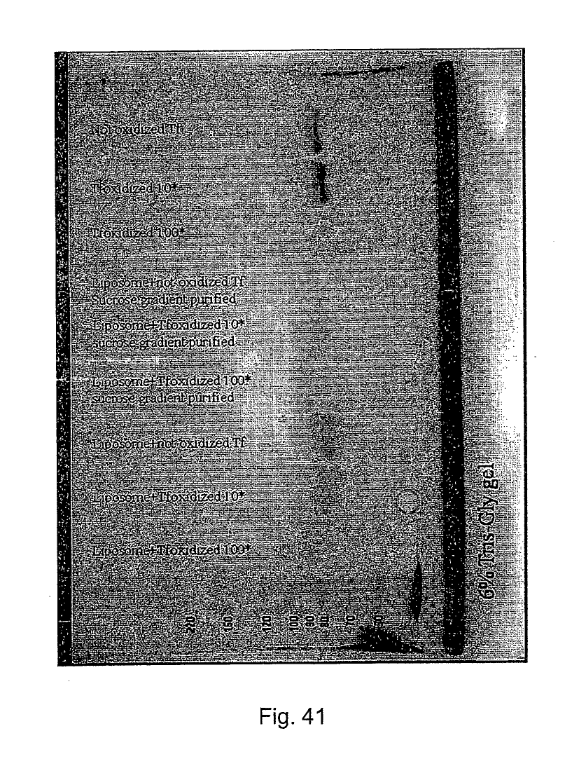

FIG. 40—Top—HPLC of DSPC/CHOUCPA liposome and non-oxidised transferrin; Middle—HPLC of DSPC/CHOUCPA liposome and transferrin oxidised with 10 eq of NalO₄; Bottom—HPLC of DSPC/CHOUCPA liposome and transferrin oxidised with 100 eq of NalO₄. In all three case, the liposome fraction was purified using an inverted sucrose gradient.

FIG. 41—Protein gel comparing the different coupling conditions, before and after sucrose separation, for DSPC/CHOL/CPA liposome and transferrin.

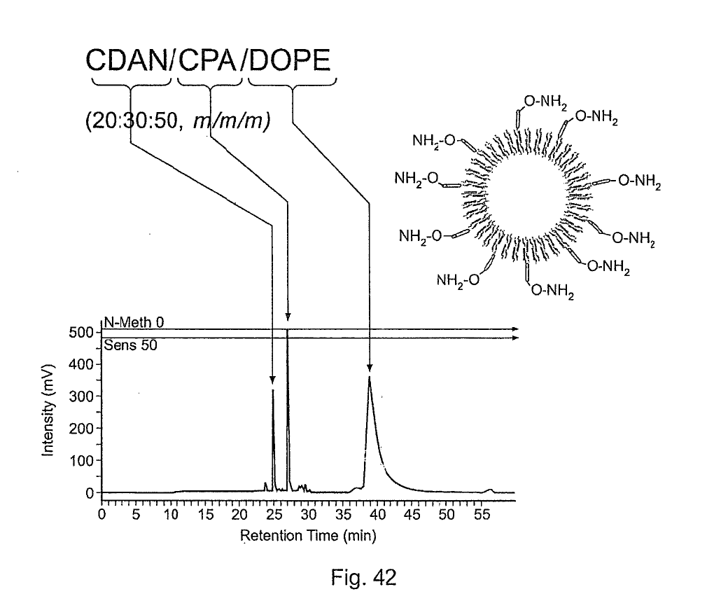

FIG. 42—HPLC of CDAN/CPA/DOPE cationic liposomes with non-oxidised antibody.

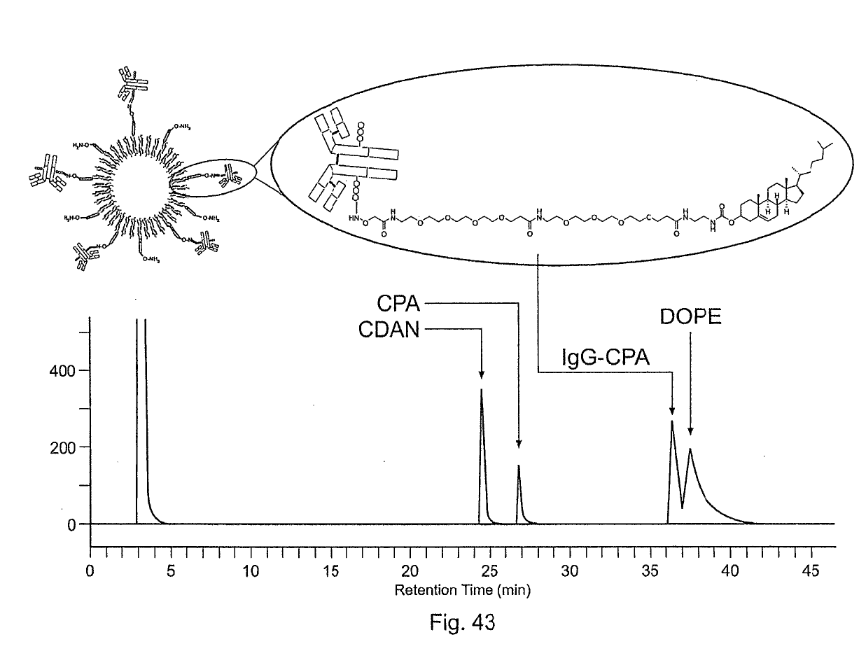

FIG. 43—HPLC of CDAN/CPA/DOPE cationic liposomes with oxidised antibody.

The present invention will now be described in further detail in the following examples.

### EXAMPLES

### Experimental Section

Synthesis of Neoglycolipids

General: ¹H NMR spectra were recorded at ambient temperature on either Brucker DRX400, DRX300, Advance Brucker 400 Ultrashield™ or Jeol GX-270Q spectrometers, with residual nonisotopicaly labeled solvent (e.g. CHCl3, δ_{N}=7.26) as an internal reference (s=singlet, d=doublet, t=triplet, q=quartet, quin=quintet, br=broad singlet). ¹³C-NMR spectra were recorded on the same range of spectrometers at 100, 75 and 68.5 MHz respectively, also with residual nonisotopicaly labelled solvent (e.g. CHCl₃, δ_{C}=77.2) as an internal reference. Infrared Spectra were recorded on Jasco FT/IR 620 using NaCI plates and Mass spectra (Positive ions electrospray) were recorded using Bruker Esquire 3000, VG-7070B or JEOL SX-102 instruments. Chromatography refers to flash column chromatography, which was performed throughout on Merck-Kieselgel 60 (230-400 mesh) with convenient solvent. Thin layer chromatography (Tlc) was performed on pre-coated Merck-Kieselgel 60 F254 aluminium backed plated and revealed with ultraviolet light, iodine, acidic ammonium molybdate(IV), acidic ethanolic vanilin, or other agents as appropriate. Neoglycolipids purity was assessed using analytical high-pressure liquid chromatography (HPLC) on a Hitachi system using a Purospher® RP-18 endcapped column (5 μm). Elution was performed at an isocratic flow rate of 1 mL/min with CH₃CN/H₂O (60:40) and fraction were detected at 205 nm wavelength before collection and Mass Analysis. Other analytical HPLC (Hitachi-LaChrom L-7150 pump system equipped with a Polymer Laboratories PL-ELS 1000 evaporative light scattering detector) was conducted on a Vydac C4 peptide column with gradient 0.1% aqueous TFA to 100% acetonitrile (0.1% TFA) [0-15 min.], then 100% acetonitrile (0.1% TFA) [15-25 min], then 100% methanol [25-45 min]. Protein fractions were analyzed on a precast 4-20% tris-glycine gel (Invitrogen, Carlsbad, Calif.). The proteins were visualized by staining for one hour with EZBlue™ Gel Staining Reagent followed by destaining overnight with deionized water. For some flash column chromatography a special eluent mixture was used i.e. Eluent A=CH₂Cl₂ 77%: MeOH 20%: H₂O 3%; Eluent B=CH₂Cl₂ 77%: MeOH 20%: ammonia (35% in water) 3%. Boc-amino-oxyacetic acid was obtained from Novabiochem (CN Biosciences, UK), PEG₂₀₀₀ bis-propionaldehyde™ was purchased from Sunbio (Korea), all other chemicals were purchased from Sigma Aldrich (Dorset, UK) unless otherwise stated. Dried CH2Cl2 was distilled with phosphorous pentoxide before use. All other dry solvents and chemicals were purchased from Sigma-Aldrich Company LTD (Poole, Dorset, UK) or BDH Laboratory Supplies (Poole, UK). HPLC-grade acetonitrile was purchased from Fisher Chemicals (Leicester, UK) and other HPLC-grade solvents from BDH Laboratory Supplies (Poole, UK).

Abbreviations: Boc: tert-butoxycarbonyl; br: broad; Chol: cholesteryl; DCM: dichloromethane; DIEA: diisopropylethylamine; DMAP: 4-(dimethylamino)pyridine DMF: N,N-dimethyl formamide; DMPC: Dimyristoylphosphatidylcholine; DMSO: dimethyl sulfoxide; DSPE: L-a-disteroyl phosphatidylethanolamine; HBTU: 2-(1H-benzotriazole-1-yl)-1,1,3,3-tetramethyluronium hexafluorophosphate; TFA: trifluoroacetic acid; THF: tetrahydrofuran.

2-(Cholesteryloxycarbonyl)aminoethanol (2): A solution of cholesteryl chloroformate (99.89 g, 0.218 mol) in CH₂Cl₂ (600 mL) was added to a stirred solution of 2-aminoethanol (29.5 mL, 0.489 mol, 2.2 equiv) in CH₂Cl₂ (450 mL) at 0° C. over a period of 2 hours. The reaction was allowed to warm to room temperature and stirring continued for a further 14 h. The reaction mixture was washed with saturated NaHCO₃ (2*200 mL), water (2*200 mL), dried (MgSO₄) and the solvents removed under reduced. The solid obtained was recrystallised (CH₂Cl₂/MeOH) to give 2 as a white solid. Yield: 99.67 g (97%); m.p.: 180° C.; R_{f}=0.26 (acetone/ether 1:9); IR (CH₂Cl₂): νₘₐₓ=3353, 2942, 2870, 1693, 1674, 1562, 1467, 1382, 1264 cm⁻¹; ¹H NMR (270 MHz, CDCl₃): δ=5.35 (d, J=6.5 Hz, 1H, H6′), 5.25-5.29 (m, 1H, NH), 4.42-4.57 (1H, m, H3′), 3.70-3.62 (m, 2H, H1), 3.25-3.35 (m, 2H, H2), 3.12 (s, 1H, OH), 2.28-2.38 (m, 2H, H4′), 1.77-2.03 (m, 5H, H2′, H7′, H8′), 1.59-0.96 (m, 21H, H1′, H9′, H11′, H12′, H14′-H17′, H22′-H25′), 1 (3H, s, H-19′), 0.9(d, J=6.5 Hz, 3H, H21′), 0.87 (d, J=6.5 Hz, 6H, H26′&H27′) and .67 (s, 3H, H18′); MS (FAB⁺): m/z=496 [M+Na]⁺, 474 [M+H]⁺, 369[Chol]⁺, 255, 175, 145, 105, 95, 81, 43.

2-[(Cholesteryloxycarbonyl)amino]ethyl methanesulfonate (3): To a solution of 2 (25 g, 52.3 mmol) and triethylamine (22 mL, 0.16 mol, 3 equiv) in CH₂Cl₂ (500 mL) at 0° C., was added dropwise a solution of methanesulfonyl chloride (10.5 mL, 0.13 mol, 2.5 equiv). The reaction mixture was allowed to warm at room temperature and stirred for 1h30. After Tlc analysis has indicated that the reaction had gone to completion, ice was added to quench the reaction. The reaction mixture was added to saturated aqueous NH₄Cl (600 mL), and extracted with ether (3*300 mL). The combined organic layers were washed with water (2*300 mL), brine (250 mL) and dried (Na₂SO₄). The solvent was remove under reduced pressure to give a white solid, which on purification by chromatography (ether) gave 3. Yield: 28.3 g (98%); IR (CH₂Cl₂): νₘₐₓ=3453, 3342, 1716, 1531, 1377, 1137 & 798 cm⁻¹; ¹H NMR (270 MHz, CDCl₃): δ=5.34 (d, J=6.5 Hz, 1H, H6′), 5-5.1 (m, 1H, NH), 4.41-4.53 (1H, m, H3′), 4.29-4.25 (t, J=5 Hz, 2H, H1), 3.47-3.52 (m, 2H, H2), 3.01 (s, 3H, H3), 2.24-2.36 (m, 2H, H4′), 1.74-2 (m, 5H, H2′, H7′, H8′), 0.9-1.6 (m, 21H, H1′, H9′, H11′, H12′, H14′-H17′, H22′-H25′), 0.98 (3H, s, H-19′), 0.84(d, J=6.5 Hz, 3H, H21′), 0.83 (d, J=6.5 Hz, 6H, H26′&H27′) and 0.65 (s, 3H, H18′); MS (FAB⁺): m/z=1104[2M+H]⁺, 574 [M+Na]⁺, 552 [M+H]⁺, 369[Chol]⁺, 255, 175, 145, 95, 81.

4-aza-N⁶(cholesteryloxycarbonylamino) hexanol (4): To a stirred solution of 3 (28.3 g, 51 mmol) dissolved in a minimum amount of THF, was added amino-propanol (160 mL, 2 mol, 39 equiv). Once Tlc indicated reaction completion (12 h), CHCl₃ (350 mL) and K₂CO₃ (20 g) were added and the solution was vigorously stirred for 30 min. The suspension was then filtered through a short pad of Celite®, washing thoroughly with CHCl₃. This was washed with a saturated solution of Sodium Hydrogenocarbonate and dried (Na₂CO₃). The solvent was removed to give 4 as a white solid. Yield: 26.1 g (96%); IR (CH₂Cl₂): νₘₐₓ=3350-3210, 2937, 2850, 1531, 1460, 1380, 1220, 1120, 1040 cm⁻¹; ¹H NMR (270 MHz, CDCl₃): δ=5.33-5.35 (m, 1H, H6′), 4.92-4.96 (m, 1H, NH), 4.42-4.51 (1H, m, H3′), 3.7-3.83. (m, 2H, H5), 3.23-3.29 (m, 2H, H1), 2.73-2.57 (m, 6H, H2, H3, H4), 2.2-2.36 (m, 2H, H4′), 1.7-2 (m, 5H, H2′, H7′, H8′), 0.85-1.58 (m, 21H, H1′, H9′, H11′, H12′, H14′-H17′, H22′-H25′), 0.98 (3H, s, H-19′), 0.84 (d, J=6.5 Hz, 3H, H21′), 0.8 (d, J=6.5 Hz, 6H, H26′&H27′) and 0.61 (s, 3H, H18′); MS (FAB⁺): m/z =543 [M+Na]⁺, 530 [M+H]⁺, 485 [M-CO₂]⁺, 369[Chol]⁺, 144 [M-Ochol]⁺, 69.55.

4-aza-(Boc)-N⁶(cholesteryloxycarbonyl amino) hexanol (5): To a solution of 4 (26.1 g, 49 mmol), was added Et₃N (8.3 mL, 1.1 equiv) and Boc₂O (10.7 g, 1 equiv) in CH₂Cl₂ (200 mL) and the resulting solution followed by tlc. On completion, the reaction mixture was poured into NH₄Cl (100 mL), and was washed with water and dried (Na₂SO₄). The solvent was removed in vacuo to give the white solid 5. The solvent was remove under reduced pressure to give a white solid, which on purification by chromatography (CH₂Cl₂/MeOH/NH₃ 92:7:1) gave 3. Yield (27.9 g, 90%); IR (CH₂Cl₂): νₘₐₓ=3352, 3054, 2937, 1675, 1530, 1455, 1380, 1220, 1120; ¹H NMR (270 MHz, CDCl₃): δ=5.33-5.35 (m, 1H, H6′), 4.86 (m, 1H, NH), 4.42-4.5 (1H, m, H3′), 3.62-3.7 (m, 2H, H5), 3.27-3.38 (m, 6H, H1, H2, H3), 2.18-2.33 (m, 2H, H4′), 1.73-2 (m, 5H, H2′, H7′, H8′), 1.45 (s, 9H, Boc), 1-1.65 (m, 23H, H4, H1′, H9′, H11′, H12′, H14′-H17′, H22′-H25′), 0.97 (3H, s, H-19′), 0.93 (d, J=6.5 Hz, 3H, H21′), 0.8 (d, J=6.5 Hz, 6H, H26′&H27′) and 0.65 (s, 3H, H18′); MS (FAB⁺): m/z=654 [M+Na]⁺, 543 [M-Boc]⁺, 369[Chol]⁺, 145, 121, 95, 69,57.

4-aza-(Boc)-N⁶(cholesteryloxycarbonylamino) hexyl methane-sulfonate (6): This experiment was carried out in a similar way as the preparation of 2-[(Cholesteryloxycarbonyl)amino]ethyl methanesulfonate 3 on 44 mmol scale giving 6. Yield (28 g, 90%); IR (CH₂Cl₂): νₘₐₓ=3305, 2980, 2900, 2865, 1675, 1530, 1455, 1350, 1150; ¹H NMR (270 MHz, CDCl₃): δ=5.33-5.35 (m, 1H, H6′), 4.86 (m, 1H, NH), 4.35-4.55 (m, 1H, H3′), 4.22 (t, 2H, J=6.5 Hz, H5), 3.2-3.4 (m, 6H, H1, H2, H3), 3.01 (s, 3H, H6), 2.15-2.33 (m, 2H, H4′), 1.73-2 (m, 5H, H2′, H7′, H8′), 1.44 (s, 9H, Boc), 1-1.67 (m, 23H, H4, H1′, H9′, H11′, H12′, H14′-H17′, H22′-H25′), 0.97 (3H, s, H-19′), 0.94 (d, J=6.5 Hz, 3H, H21′), 0.8 (d, J=6.5 Hz, 6H, H26′&H27′) and 0.65 (s, 3H, H18′); MS (FAB⁺): m/z=722 [M+Na]⁺, 609 [M-Boc]⁺, 369[Chol]⁺, 145, 121, 95, 69, 55.

4-aza-(Boc)-N⁶(cholesteryloxycarbonylamino) hexanamine (7): To 6 (25 g, 35 mmol), sodium azide (11.49, 175.7 mmol, 5 equiv), and sodium iodine (5 g, 35 mmol, 1 equiv) under nitrogen was added anhydrous DMF (200 mL), with stirring. Equipped with a reflux condenser, heating at 80° C. for 2 h resulted in completion of reaction. The reaction mixture was allowed to cool to room temperature, the DMF removed under reduced pressure and the residue dissolved in EtOAc. This was washed with water (2*100 mL), brine (100 mL) and dried (Na₂SO₄) to give after purification by chromatography (hexane/ether 1:1) 7 as a white solid. Yield (22 g, 95%); ¹H NMR (270 MHz, CDCl₃): δ=5.34-5.36 (m, 1H, H6′), 4.35-4.55 (m, 1H, H3′), 4.25 (t, 2H, J=6.5 Hz, H5), 3.2-3.5 (m, 6H, H1, H2, H3), 2.25-2.33 (m, 2H, H4′), 1.7-2.05 (m, 5H, H2′, H7′, H8′), 1.45 (s, 9H, Boc), 1-1.72 (m, 23H, H4, H1′, H9′, H11′, H12′, H14′-H17′, H22′-H25′), 0.98 (3H, s, H-19′), 0.94 (d, J=6.5 Hz, 3H, H21′), 0.83 (d, J=6.5 Hz, 6H, H26′&H27′) and 0.64 (s, 3H, H18′); MS (FAB′): m/z=568 [M+Na-Boc]⁺, 556 [M-Boc]⁺, 369[Chol]⁺, 145, 121, 95, 69,57.

4-aza-(Boc)-N⁶(cholesteryloxycarbonylamino) hexylamine (8): To a round bottomed flask charged with 7 (22.75 g, 34.6 mmol) in THF (230 mL) was added trimethylphosphine in THF (1 M, 40 mL, 1.15 equiv), and the reaction was monitored by tic. On the completion the reaction was stirred with water (3 mL) and aqueous ammonia (3 mL) for 1 h and the solvent was remove under reduce pressure. After chromatography (CH₂Cl₂/MeOH/NH₃ 92:7:1 to 75:22:3) 8 was obtained as a white crystal. Yield (19.1 g, 88%); IR (CH₂Cl₂): νₘₐₓ=3689, 3456, 3155, 2948, 2907, 2869, 2253, 1793, 1709, 1512, 1468, 1381, 1168; ¹H NMR (270 MHz, CDCl₃): δ=5.32-5.35 (m, 1H, H6′), 4.35-4.51 (m, 1H, H3′), 3.45-3.05 (m, 8H, H1, H2, H3, H5), 2.18-2.4 (m, 2H, H4′), 1.8-2.1 (m, 5H, H2′, H7′, H8′), 1.46 (s, 9H, Boc), 1.01-1.72 (m, 23H, H4, H1′, H9′, H11′, H12′, H14′-H17′, H22′-H25′), 0.97 (3H, s, H-19′), 0.85 (d, J=6.5 Hz, 3H, H21′), 0.82 (d, J=6.5 Hz, 6H, H26′&H27′) and 0.64 (s, 3H, H18′); MS (FAB′): m/z=630 [M+H]⁺, 530 [M-Boc]⁺, 369[Chol]⁺, 145, 121, 95, 69, 57.

(Boc)aminooxyacetic acid (9): O-(Carboxymethyl)hydroxylamine hemihydrochloride (1.16 g, 5.3 mmol) was dissolved in CH₂CI₂ (40 mL) and the pH was adjusted to 9 by addition of triethylamine (3 mL). Then di-tert-butyl dicarbonate (2.36 g, 10.6 mmol, 2.0 equiv) was added and the mixture was stirred at room temperature until tic indicated completion of reaction. The pH was lowered to 3 by addition of diluted HCl. The reaction mixture was partitioned between saturated aqueous NH₄Cl (20 mL) and CH₂Cl₂ (30 mL). The aqueous phase was extracted with CH₂Cl₂ (3×100 mL). The combined organic extracts were washed with H₂O (2×100 mL) and dried (Na₂SO₄). The solvent was removed in vacuo to afford 9 as a white solid. Yield (1.86 g, 97%); IR (CH₂Cl₂): νₘₐₓ=3373, 2983, 2574, 2461, 1724, 1413, 1369, 1235; ¹H NMR (270 MHz, CDCl₃): δ=4.48 (s, 2H, CH₂), 1.48 (s, 9H, Boc); MS (FAB′): m/z=214 [M+Na]⁺, 192 [M+H]⁺, 135, 123, 109, 69.

(Boc)aminooxy compound (10): N-hydroxysuccinimide (0.36 g, 3.13 mmol, 1 equiv), 9 (0.6 g, 3.13 mmol, 1 equiv), and N,N′-dicyclohexylcarbodiimide (0.68 g, 3.13 mmol, 1 equiv) were dissolved in EtOAc (90 mL), and the heterogeneous mixture was allowed to stir at room temperature overnight. The mixture was then filtered through a pad of Celite® to remove the dicyclohexylurea, which was formed as a white precipitate (rinsed with 60 mL of EtOAc), and added to a solution of 8 (1.97 g, 3.13 mmol, 1 equiv) in THF (10 mL). A pH of 8 was maintained for this heterogeneous reaction by addition of triethylamine (6 mL). The resulting mixture was allowed to stir at room temperature overnight. On completion the mixture was filtered and the solvent was removed under reduced pressure to give after purification by flash-chromatography (CH₂Cl₂/MeOH/NH₃ 92:7:1) 10 as a white solid. Yield (2.3 g, 90%); ¹H NMR (270 MHz, CDCl₃): δ=5.33-5.35 (m, 1H, H6′), 4.4-4.52 (m, 1H, H3′), 4.3 (s, 2H, H90, 3.2-3.42 (m, 8H, H1, H2, H4, H6), 2.23-2.35 (m, 2H, H4′), 1.7-2.1 (m, 7H, H2′, H7′, H8′, H5), 1.44-1.46 (m, 18H, 2 Boc), 1-1.73 (m, 21H, H1′, H9′, H11′, H12′, H14′-H17′, H22′-H25′), 0.98 (3H, s, H-19′), 0.85 (d, J=6.5 Hz, 3H, H21′), 0.83 (d, J=6.5 Hz, 6H, H26′&H27′) and 0.65 (s, 3H, H18′); MS (FAB⁺): m/z=803 [M+H]⁺, 703 [M-Boc]⁺, 647, 603 [M-2Boc]⁺, 369, 279, 255, 235, 204, 145, 95, 69.

Hydroxylamine (11): To a solution 10 (1.1 g, 1.36 mmol, 1 equiv) in CH₂Cl₂ (10 mL) was added TFA (2 mL, 20.4 mmol, 15 equiv) at 0° C. The solution was allowed to stir at room temperature for 5 hours. On completion toluene was added to azeotrope TFA from the reaction mixture. The solvents were removed in vacuo to afford after purification by chromatography (CH₂Cl₂/MeOH/NH₃ 92:7:1 to 75:22:3) 11 as a white solid (709 mg, Yield: 86%); IR (CHCl₃): νₘₐₓ=3306, 2948, 2850, 2246, 1698, 1647, 1541, 1467, 1253, 1133; ¹H NMR (270 MHz, CDCl₃): δ=5.26-5.4 (m, 1H, H6′), 4.4-4.52 (m, 1H, H3′), 4.12 (s, 2H, H9), 3.34-3.41 (m, 2H, H2), 3.15-3.3 (m, 2H, H4), 2.6-2.74 (m, 4H, Hi & H6), 2.14-2.39 (m, 2H, H4′), 1.62-2.1 (m, 7H, H2′, H7′, H8′, H5), 1.02-1.6 (m, 21H, H1′, H9′, H11′, H12′, H14′-H17′, H22′-H25′), 0.96 (3H, s, H-19′), 0.86 (d, J=6.5 Hz, 3H, H21′), 0.83 (d, J=6.5 Hz, 6H, H26′&H27′) and 0.66 (s, 3H, H18′); MS (FAB⁺): m/z=603 [M+H]⁺, 369[Chol]⁺, 160, 137, 109, 95, 81, 69, 55.

Mannosyl compound (12a): A solution of D-mannose (266 mg, 4.8 mmol) in Acetic aqueous Buffer (sodium acetate/acetic acid 0.1 M, pH 4, 7mL) and a solution of 11 (290 mg, 0.48 mmol, 10 equiv) in DMF (7 mL) was mixed and stirred for 3 days at room temperature. The solvent was removed in vacuo by freeze drying and chromatography (CH₂Cl₂/MeOH/NH₃ 75:22:3) afforded the product 21 a white solid (233 mg, Yield : 65%). The purity was further confirmed by HPLC. The final product contained of the β-pyranose (82%) form and α-pyranose (18%) form that were not isolated but characterized in the mixture. MS (FAB⁺): m/z=765 [M+H]⁺, 787 [M+Na]⁺, 397, 369[Chol]⁺, 322, 240, 121, 109, 95, 81, 69, 57. β-pyranose form ¹H NMR (400 MHz, CD₃OD/CDCl3 [75/25]): δ=7.64-7.62 (d, ³J₁ₐ₋₂ₐ=7 Hz, 1H, H1a), 5.35-5.36 (m, 1H, H6′), 4.45-4.5 (s, 2H, H9), 4.35-4.5 (m, 1H, H3′), 4.19-4.24 (dd, 1H, H2ₐ, ³J₁ₐ₋₂ₐ=7.4 Hz, ³J₂ₐ₋₃ₐ=7.7 Hz), 3.81-3.9 (m, 1H, H3a), 3.73-3.8 (m, 2H, H4a, H6ₐₓa), 3.63-3.71 (m, 2H, H5a, H_{eq}6a), 3.34-3.42 (m, 2H, H2), 3.27-3.30 (m, 2H, H4), 3-3.08 (m, 2H, H1), 2.9-2.98 (m, 2H, H6), 2.25-2.35 (m, 2H, H4′), 1.78-2.07 (m, 7H, H2′, H7′, H8′, H5), 1.03-1.65 (m, 21H, H1′, H9′, H11′, H12′, H14′-H17′, H22′-H25′), 1.01 (3H, s, H-19′), 0.91 (d, J=6.5 Hz, 3H, H21′), 0.85 (d, J=6.5 Hz, 6H, H26′&H27′) and 0.69 (s, 3H, H18′); ¹³C NMR (400 MHz, CDCl₃/CD₃OD [25/75]): 12.33 (C18′), 19.20 (C21′), 19.74 (C19′), 21.91 (C11′), 22.91 (C27′), 23.17 (C26′), 24.67 (C23′), 25.07 (C15′), 27.37 (C5), 28.85 (C25′), 28.96 (C2′), 29.07 (C12′), 32.76 (C7′), 32.87 (C8′), 36,38 (C2), 36.78 (C20′), 37.09 (C1) 37.76 (C22′),37.95 (C1′), 38.4 (C4), 39.36 (C4′), 40.41 (C24′), 40.76 (C16′), 46.16 (C6), 51.19 (C9′), 57.19 (C17′), 57.75 (C14′), 64.62 (C6a), 70.19 (C2a), 70.58 (C4a), 72.12 (C3a), 72.37 (C5a), 73.11 (C9), 75.91 (C3′), 123.39 (C6′), 140.72 (C5′), 155.02 (C1a), 158.69 (NHCOOChol), 173.1 (C8); α-pyranose form : identical data except, ¹H NMR (400 MHz, CD₃OD/CDCl3 [75/25]): δ=6.90-6.88 (d, ³J₁ₐ₋₂ₐ=7 Hz, 1H, H1a), 5-5.05 (dd, 1H, H2a, ³J₁ₐ₋₂ₐ=7.3 Hz, ³J₂ₐ₋₃₁=7.6 Hz); ¹³C NMR (400 MHz, CDCl₃/CD₃OD [25/75]): 65.33 (C2a), 155.79 (C1a). ¹H NMR (400, CD₃OD/CDCl₃ [75/25]): (m, 1H, H3′) missing, underneath solvent peak; confirmed by ¹H NMR (300 MHz, DMSO): δ=4.67-4.82 (m, 1H, H3′). ¹³C NMR (400 MHz, CDCl₃/CD₃OD [25/75]): C1 missing, underneath MeOH peak confirmed by ¹H/¹³C correlation at 400 MHz, around 49. Proton resonance assignments were confirmed using ¹H gradient type DQF-COSY and TOCSY; ¹H/¹³C correlation and DEPT 135 were used to assign unambiguously the carbon resonances. α pyrannose form gave ¹J¹³C1a-H1a=177 Hz and β pyrannose form gave ¹J¹³C1a-H1a=167 Hz. ¹H phase-sensitive NOESY confirmed conformation.

Glucosyl compound (12b): This was prepared with a solution of D-glucose (150 mg, 0.82 mmol) and 11 (100 mg, 0.16 mmol) in a similar way to the preparation of 12a, stirred for 1 day and purified by chromatography (CH₂Cl₂/MeOH/NH₃ 75:22:3) to afford the product 12b as a white solid (103 mg, Yield: 82%). The purity was further confirmed by HPLC. The final product contained of the α-pyranose (11%) anomer and β-pyranose (89%) anomer that were not isolated but characterized in the mixture. (FAB⁺): m/z=765 [M+H]⁺, 787 [M+Na]⁺, 391, 369 [Chol]⁺, 309, 290, 171, 152, 135, 123, 109, 95, 81, 69; β-pyranose form. (300 MHz, CDCl₃/CD₃OD [90/10]): δ=7.53-7.56 (d, J=5.6 Hz, 1H, H1a), 5.26-5.36 (m, 1H, H6′), 4.2-4.45 (m, 3H, H9, H3′), 4.05-4.15 (m, 1H, H2a), 3.45-3.85 (m, 5H, H6a, H3a, H5a, H4a), 2.9-3.4 (m, H2, H4, MeOH), 2.9-3.15 (m, 4H, H1, H6), 2.15-2.3 (m, 2H, H4′), 1.65-2 (m, 5H, H2′, H7′, H8′), 0.95-1.55 (m, 23H, H5, H1′, H9′, H11′, H12′, H14′-H17′, H22′-H25′), 0.93 (3H, s, H-19′), 0.84 (d, J=6.5 Hz, 3H, H21′), 0.78 (d, J=6.5 Hz, 6H, H26′&H27′) and 0.62 (s, 3H, H18′); α-pyranose form: identical data except, ¹H NMR (300 MHz, CDCl₃/CD₃OD [90/10]): δ=7.22-7.24 (d, J=6,61 Hz, 1H, H1a), 4.95-5.07 (m, 1H, H2a); ¹H NMR (300 MHz, CD₃OD): (m, 1H, H3′) missing, presumably underneath solvent peak; confirmed by ¹H NMR (300 MHz, DMSO): δ=4.7-4.86 (m, 1H, H3′)

Galactosyl compound (12c): This was prepared with a solution of D-galactose (50 mg, 0.27 mmol) and 11 (40 mg, 0.066 mmol in a similar way to the preparation of 12a, stirred for 1 day and purified by chromatography (CH₂Cl₂/MeOH/NH₃ 75:22:3) to afford the product 12c as a white solid (35 mg, Yield: 70%). The purity was further confirmed by HPLC. The final product contained of the a-pyranose (15%) form and β-pyranose (85%) form that were not isolated but characterized in the mixture. MS (FAB⁺): m/z=765 [M+H]⁺, 588, 391, 369 [Chol]⁺, 322, 290, 165, 152, 135, 121, 109, 95, 81, 69; β-pyranose form. ¹H NMR (270 MHz, DMSO): δ=7.78-7.82 (m, 1H, NHCO of C8), 7.55-7.58 (d, J=7.2 Hz, 1H, H1a), 6.95-7.1 (m, 1H, NHCOOChol), 5.25-5.37 (m, 1H, H6′), 4.2-4.43 (m, 3H, H9, H3′), 3.2-3.9 (m, H2a, H6a, H3a, H5a, H4a, OH), 2.9-3.18 (m, 4H, H2, H4), 2.4-2.65 (m, 4H, H1, H6), 2.15-2.3 (m, 2H, H4′), 1.67-2 (m, 5H, H2′, H7′, H8′), 0.92-1.6 (m, 23H, H5, H1′, H9′, H11′, H12′, H14′-H17′, H22′-H25′), 0.96 (3H, s, H-19′), 0.89 (d, J=6.5 Hz, 3H, H21′), 0.84 (d, J=6.5 Hz, 6H, H26′&H27′) and 0.65 (s, 3H, H18′). α-pyranose form: identical data except, ¹H NMR (270 MHz, DMSO): 6.86-6.88 (d, J=6 Hz, 1H, H1a) Glucuronic compound (12d): This was prepared with a solution of D-glucuronic acid, sodium salt monohydrate (30 mg, 0.128 mmol, 1.5 equiv) and 11 (50 mg, 0.08 mmol) in a similar way to the preparation of 12a, stirred for 1 day, purified by chromatography (CH₂Cl₂/MeOH/NH₃ 75:22:3) to afford the sodium salt of 12d as a white solid (41 mg, Yield: 60%). The purity was further confirmed by HPLC. The final product contained of the α-pyranose (85%) form and β-pyranose (15%) form that were not isolated but characterized in the mixture. MS (FAB⁺): m/z=779 [M+H]⁺, 733, 588, 411, 369[Chol]⁺, 336, 290, 240, 214, 159, 145, 135, 121, 109, 95, 81, 69, 55. β-pyranose form. ¹H NMR (300 MHz, CDCl₃/CD₃OD [75/25]): δ=7.51-7.53 (d, J=5.9 Hz, 1H, H1a), 5.25-5.33 (m, 1H, H6′), 4.2-4.45 (m, 3H, H9, H3′), 3.8-4.1 (m, 3H, H2a, H3a, H4a), 3.6-3.75 (m, 1H, H5a), 3.2-3.55 (m, H2, H4, MeOH), 2.7-3.15 (m, 4H, H1, H6), 2.18-2.32 (m, 2H, H4′), 1.62-2 (m, 5H, H2′, H7′, H8′), 0.9-1.6 (m, 23H, H5, H1′, H9′, H11′, H12′, H14′-H17′, H22′-H25′), 0.93 (3H, s, H-19′), 0.83 (d, J=6.5 Hz, 3H, H21′), 0.77 (d, J=6.5 Hz, 6H, H26′&H27′) and 0.6 (s, 3H, H18′) ); α-pyranose form: identical data except, ¹H NMR (300 MHz, CD₃OD): δ=7.22-7.24 (d, J=6.3 Hz, 1H, H1a), 5-5.1 (m, 1H, H2a).

β-D-lactosyl compound (12e): A solution of β-D-Lactose, containing 25-30% of α (1.13 g, 3.3 mmol) and 11 (200 mg, 0.33 mmol) in 14 mL of DMF/Acetic aqueous Buffer was stirred for 4 days at room temperature. The solvent was removed in vacuo by freeze-drying and chromatography (CH₂Cl₂/MeOH/NH₃ 75:22:3) afforded the product 12e as a white solid (145 mg, Yield: 47%). The purity was further confirmed by HPLC. The final product contained of the a-pyranose (15%) form and β-pyranose (85%) form (containing itself around 25% of α lactose) that were not isolated but characterized in the mixture. MS (FAB⁺): m/z=927 [M+H]⁺, 588, 482, 369[Chol]⁺, 290, 243, 216, 178, 152, 135, 121, 109, 95, 81, 69, 55; β0pyranose form. ¹H NMR (400 MHz, CDCl₃/CD₃OD [20/80]): δ=7.69-7.71 (d, ³J₁ₐ₋₂ₐ=5.8 Hz, 1H, H1a of βlactose), 7.66-7.68 (d, ³J₁ₐ₋₂ₐ=6.2 Hz, 1H, H1a of α lactose), 5.35-5.37 (m, 1H, H6′), 4.374.6 (m, 4H, H9, H3′, H2a), 4.2-4.37 (m, 1H, H1b), 3.65-4.05 (m, 7 H, H3a, H4a, H5a, H4b, H5b, H6b), 3.25-3.6 (m, 8H, H2, H4, H6a, H2b, H3b, MeOH), 3-3.2 (m, 4H, H1, H6), 2.25-2.42 (m, 2H, H4′), 1.8-2.15 (m, 5H, H2′, H7′, H8′), 1-1.65 (m, 23H, H5, H1′, H9′, H11′, H12′, H14′-H17′, H22′-H25′), 1.01 (3H, s, H-19′), 0.91 (d, J=6.5 Hz, 3H, H21′), 0.85 (d, J=6.5 Hz, 6H, H26′&H27′) and 0.69 (s, 3H, H18′); ¹³C NMR (400 MHz, CDCl₃/CD₃OD [20/80]): ¹³C NMR (400 MHz, CDCl₃/CD₃OD [20/80]): 12.32 (C18′),19.2 (C21′), 19.76 (C19′), 21.94 (C11′), 22.91 (C27′), 23.17 (C26′), 24.7 (C23′), 25.1 (C15′), 27.22 (C5), 28.89 (C25′), 29 (C2′), 29.1 (C12′), 32.8 (C7′), 32.92 (C8′), 36.29 (C22′), 36.81 (C10′), 37.12 (C1′), 37.99 (C6), 38.11 (C1), 39.48 (C2), 40.45 (C24′), 40.80 (C16′), 46.13 (C4′), 51.23 (C9′), 57.22 (C17′), 57.80 (C14′), 62.41 (C6a), 63.4 (C6a), 70.02 (C5b), 70.63 (C2a), 72.8 (C3a), 73 (C3′), 73.18 (C9), 74,75 (C2b), 76.8 (C3a), 81 (C4b), 92.39 (C1b), 105.2 (C3′), 123.42 (C6′), 140.72 (C5′), 154.8 (C1a), 156.2 (NHCOOChol), 173.17 (C8), α-pyranose form : identical data except, ¹H NMR (400 MHz, CD₃OD/CDCl3 [80/20]): δ_{H}=7.04-7.05 (d, ³J₁ₐ₋₂ₐ=5.6 Hz, 1H, H1a), 5.05-5.07 (m, 1H, H2a), 4.09-4.11 (m, 1H, H3a); ¹H NMR (270 MHz, CD₃OD): (m, 1H, H3′) missing, presumably underneath solvent peak; confirmed by ¹H NMR (300 MHz, DMSO): δ=4.7-4.85 (m, 1H, H3′). Proton resonance assignments were confirmed using ¹H gradient type DQF-COSY and TOCSY; ¹H/¹³C correlation and DEPT 135 were used to assign unambiguously the carbon resonances. ¹H phase-sensitive NOESY confirmed conformation. Maltosyl compound (12f): This was prepared with a solution of D Maltose monohydrate (30 mg, 1.8 mmol, 5 equiv) and 11 (100 mg, 0.16 mmol) ) in a similar way to the preparation of 12e, stirred for 1 day and purified by chromatography (CH₂Cl₂/MeOH/NH₃ 75:22:3) to afford 12f as a white solid (100 mg, Yield: 65%). The purity was further confirmed by HPLC. The final product contained of the a-pyranose (87%) form and β-pyranose (13%) form that were not isolated but characterized in the mixture. MS (FAB⁺): m/z=927 [M+H]⁺, 765, 588, 559, 484, 369[Chol]+, 322, 290, 213, 167, 161, 143, 135, 121, 109, 95, 81, 69, 55. β-pyranose form. ¹H NMR (300 MHz, CDC13/CD₃0D [80/20]): δ=7.55-7.57 (d, ³Ja-₂a =5.3 Hz, 1H, H1a), 5.3 (s, 1H, H6′), 4.85-5.02 (m, 1H, H3′), 4.09-4.22 (m, 1H, H1b), 3.57-4 (m, 7 H, H3a, H4a, H5a, H4b, H5b, H6b), 3.2-3.6 (m, 8H, H2, H4, H6a, H2b, H3b,MeOH), 2.8-3.1 (m, 4H, H1, H6), 2.1-2.36 (m, 2H, H4′), 1.6-2.05 (m, 5H, H2′, H7′, H8′), 1-1.6 (m, 23H, H5, H1′, H9′, H11′, H12′, H14′-H17′, H22′-H25′), 0.93 (3H, s, H-19′), 0.83 (d, J=6.5 Hz, 3H, H21′), 0.78 (d, J=6.5 Hz, 6H, H26′&H27′) and 0.6 (s, 3H, H18′); α-pyranose form: identical data except, ¹H NMR (300 MHz, CD₃0D/CDC13 [80/20]): & 6.92-6.94 (d, J=4.62 Hz, 1 H, H1a), 5.02-5.15 (m, 1 H, H2a), 4.04-4.08 (m,1 H, H3a) Maltotriosyl compound (12 g): This was prepared with a solution of maltotriose (246.4 mg, 0.46 mmol, 7 equiv) and 11 (40 mg, 0.066 mmol) in a similar way to the preparation of 12e, stirred for 5 days and purified by chromatography (CH₂Cl₂/MeOH/NH₃ 75:22:3) to afford 12f as a white solid (61 mg, Yield: 85%). The purity was further confirmed by HPLC. The final product contained of the a-pyranose (15%) form and β-pyranose (85 %) form that were not isolated but characterized in the mixture. MS (FAB⁺): m/z=1111 [M+Na]⁺, 1089 [M+H]⁺, 588, 423, 391, 369 [Chol]+, 240, 171, 159, 145, 121, 105, 95, 81, 69; β-pyranose form: ¹H NMR (300 MHz, CDCl3/MeOH[20/80]): δ=7.56-7.58 (d, J=6 Hz, 1H, H1a), 5.2-5.27 (m, 1H, H6′), 4.9-4.95 (m, 1H, H3′), 4.2-4.45 (m, 4H, H9, H3′, H2a), 4.05-4.2 (m, 2H, H1b, H1c), 2.95-4 (m, 21H, H2, H4, H6a, H3a, H5a, H4a, H2b-6b, H2c-6c, MeOH), 2.85-2.95 (m, 4H, H1, H6), 2.2-2.3 (m, 2H, H4′), 1.8-2.1 (m, 5H, H2′, H7′, H8′), 0.98-1.6 (m, 23H, H5, H1′, H9′, H11′, H12′, H14′-H17′, H22′-H25′), 0.94 (3H, s, H-19′), 0.84 (d, J=6.5 Hz, 3H, H21′), 0.78 (d, J=6.5 Hz, 6H, H26′&H27′) and 0.61 (s, 32 3H, H18′); α-pyranose form: identical data except, ¹H NMR (300 MHz, CDCl₃/MeOH[20/80]): δ=6.85 (d, J=5.6 Hz, 1H, H1a). Maltotetraosyl compound (12h): This was prepared with a solution of D Maltotetraose (200 mg, 0.3030 mmol) and 11 (80 mg, 0.133 mmol, stirred for 5 days and purified by chromatography (CH₂Cl₂/MeOH/NH₃ 75:22:3) to afford 12h as a white solid (67.5 mg, Yield: 41%). The purity was further confirmed by HPLC. The final product contained of the a-pyranose (15%) form and β-pyranose (85%) form that were not isolated but characterized in the mixture. MS (FAB+): m/z =1273 [M+Na]+, 1251 [M+H]+, 588, 369 [Chol]+, 159, 145, 121, 109, 95, 81, 69; HRMS (FAB+) C₅₉H₁₀₂N₄O₂₄Na: [M+Na]+calcd 1273.6782, found 1273.6821. β-pyranose form: ¹H NMR (300 MHz, CDCl₃/MeOH[20/80]): δ=7;56-7.58 (d, 1 H, H1 a), 5.15-5.25 (m, 1 H, H6′), 4.95-5.1 (m, 1H, H3′), 4.38-4.5 (m, 4H, H9, H3′, H2a), 4.04-4.22 (m, 3H, H1b, H1c, H1d), 3.1-3.95 (m, 27H, H2, H4, H6a, H3a, H5a, H4a, H2b-6b, H2c-6c, H2d-6d, MeOH), 2.85-3.1 (m, 4H, H1, H6), 2.2-2.33 (m, 2H, H4′), 1.75-2.1 (m, 5H, H2′, H7′, H8′), 1-1.6 (m, 23H, H5, H1′, H9′, H11′, H12′, H14′-H17′, H22′-H25′), 0.92 (3H, s, H-19′), 0.82 (d, J=6.5 Hz, 3H, H21′), 0.78 (d, J=6.5 Hz, 6H, H26′&H27′) and 0.68 (s, 3H, H18′); α-pyranose form: identical data except, ¹H NMR (300 MHz, CDCl₃/MeOH[20/80]): & 7 (d, 1H, H1a). Maltoheptaosyl compound (12i): This was prepared with a solution of D Maltoheptaose (100 mg, 0.08673 mmol) and 11 (30 mg, 0.0497 mmol) stirred for 7 days and purified by chromatography (CH₂Cl₂/MeOH/NH₃ 75:22:3) to afford 12i as a white solid (46mg, Yield: 53%). The purity was further confirmed by HPLC. The final product contained of the α-pyranose (15%) form and β-pyranose (85%) form that were not isolated but characterized in the mixture. MS (FAB+): m/z =1759 [M+Na]+, 1737 [M+H]⁺, 369 [Chol]+, 145, 121, 109, 95, 81. β-pyranose form: ¹H NMR (300 MHz, CDCl₃/MeOH[20/80]): δ=7.53-7.58 (d, 1H, H1a), 5.35-5.37 (m, 1H, H6′), 4.97-5.12 (m, 1H, H3′), 4.45-4.6 (m, 4H, H9, H3′, H2a), 4-4.5 (m, 6H, H1b, H1c-g), 3.1-3.9 (m, 45H, H2, H4, H6a, H3a, H5a, H4a, H2b-6b, H2c-6c, H2d-6d, H2e-6e , H2f-6f, H2 g-6 g, MeOH), 2.7-3 (m, 4H, H1, H6), 2.15-2.35 (m, 2H, H4′), 1.7-2.1 (m, 5H, H2′, H7′, H8′), 1-1.6 (m, 23H, H5, H1′, H9′, H11′, H12′, H14′-H17′, H22′-H25′), 0.94 (3H, s, H-19′) 0.84 (d, J=6.5 Hz, 3H, H21′), 0.77 (d, J=6.5 Hz, 6H, H26′&H27′) and 0.63 (s, 3H, H18′); α-pyranose form: identical data except, ¹H NMR (300 MHz, CDCl₃/MeOH[20/80]): δ=6.9 (d, 1H, H1a). 33

3-ethoxypropionaIdehyde (14): 3-ethoxypropionaldehyde diethylacetal (0.5 mL, 2.46 mmoles) was diluted in THF (2 mL) and the solution was cooled to 0° C. on an ice bath. 2 mL of a 1M aqueous solution of HCl were added and the reaction mixture was allowed to reach room temperature. The reaction was left stirring for 1 hr. The aldehyde was extracted with 2×5mL of ethyl acetate, the organic layer was dried over sodium sulphate and concentrated in vacuo to afford the aldehyde 14 as a colourless liquid. ¹H NMR (400MHz, CDCl₃) 6 =1.20 (t, 3H, J=6.8, CH₃CH₂O), 2.67 (dt, 2H, J=2, J=5.2, CH₂CH₂CH═N), 3.52 (q, 2H, J=6.8, CH₃CHO), 3.77 (t, 2H, J=6, CHCH₂CH=N), 9.81 (t, 1H, J=2, CHO). ¹³C NMR (400MHz, CDC1₃) 6 =15.03 (CH₃), 43.93 (CH₂), 64.07 (CH₂), 66.54 (CH₂), 201.28 (C=O). 3-ethoxy-propylidene-amino-oxy acetic acid (16): O-(carboxymethyl)-hydroxylamine 15 (0.01 g, 0.11 mmoles) and 3-ethoxypropion-aldehyde 14 (0.0112 g, 0.011 mmoles) were dissolved in a mixture of 1.5 ml of CDCl₃ and 0.5 mL of DMSO-d6. The reaction mixture was left to give the oxime 16. ¹H NMR (400 MHz, DMSO/CDCl3 1/3) 6 =1.08 (m, 3H, CH₃), 2.35 and 2.54 (2q, 2H, J=7.2, CH₂), 3.38 and 3.40 (2q, 2H, J=7.2, CH₂), 3.47 and 3.48 (2t, 2H, J=6, CH₂), 4.40 and 4.45 (s, 2H, CH₂), 6.71 and 7.44 (t, 1H, J=5.2, N═CH). ¹³C NMR (400 MHz, DMSO/CDC₃ 1/3) δ=14.56 (CH₃), 26.12 and 29.45 (CH₂), 65.45 and 65.49 (CH₂), 66.02 and 66.62 (CH₂), 69.46 and 69.57 (CH₂), 149.40 and 149.91 (C=N-O). 171.05 and 171.07 (C=O). Cholesteryl-oxime-lipid (19): 3-ethoxypropionaldehyde 14 (2 mg, 9.16 pmoles) and Cholesteryl-aminoxy-lipid 25 (10 mg, 9.16pmoles) were dissolved in CDCl₃ to give oxime 19. ¹H NMR (400 MHz, CDC1₃) 0.68 (s, 3 H, Chol C-18), 0.83, 0.82 (2×d, 6 H, J=6.5 and 2.0 Hz), 0.89 (d, 3 H, J=6.4, Chol C-21), 1.0 (s, 3 H, Chol C-19), 1.21 (t, 3H, J=7.2, CH₃CH₂O), 0.94-2.10 (Chol C-1, 2, 4, 7, 8, 9, 11, 12, 14, 15, 16, 17, 20, 22, 23, 25),2.32 (m, 2 H, Chol C-24), 2.5 and 2.71 (2q, 2H, J=6.4, CH₂CH=N), 3.3 (m, 2H, O(CO)NHCH₂CH₂), 3.45 (m, 2H, NHCH₂CH₂-NHCO-CH₂), 3.5 (q, 2H, J=7.2, OCH₂CH₃), 3.6 (t, 2H, J=6.4, CH₂O), 4.4 (m, 1H, Chol C-3), 4.45 (s br, 1H, NH), 4.5 and 4.6 (2s, 2H, (CO)CH ON), 4.8 (m, 1 H, Chol-O(CO)NH), 5.0 (s br, 1H, NH),5.35 (m, 1H, Chol C6), 6.72 (s br, 1H, NH), 6.88 and 7.60 (2t, 1H, J=5.4, CH═N), ¹³C NMR (100 MHz, CDC1₃) 11.8 (C-18), 15.1 (CH₃CH₂0) 18.7 (C-21), 19.1 (C-19), 21.0 (C-11),) 22.6 (C-27), 22.8 (C-26),23.8 (C-23), 24.3 (C-15), 28.0 (C-25), 28.1 (C-2), 28.2 (C-16), 30.1 (CH₂CHN), 31.8 (C-7), 31.9 (C-8), 35.8 (C-20), 36.2 (C-22), 36.6 (C-10), 37.0 (C-1), 38.5 (C-24), 39.6-39.7 ((C-12, C-4), O(CO)NHCH₂CH₂ overlapping), 40.7 (C-4), 42.3 (C-13), 34 50.0 (C-9), 56.2 (C-17), 56.7 (C-14), 66.7 (OCH₂CH₃), 67.2 (CH₂O), 72.5 (C-3), 72.8 ((CO)CH₂ONH₂), 122.6 (C-6), 139.8 (C-5), 151.4 and 151.7 (CH═NO), 158.3 (OCONH) and 171.4 (NH(CO)CH₂ONH₂).

Post-Coupling Reaction

Cholesteryl-oxime-lipid (19): Dimyristoylphosphatidylcholine (DMPC) (5 mg, 7.18 μmoles) and Cholesteryl-aminoxy-lipid 25 (4.8 mg, 8.18 μmoles) were combined in chloroform to prepare liposomes of DMPC/25 (45:55, w:w).The solution was transferred into a round bottom flask and organic solvent were removed under reduced pressure giving a thin lipid film that was dried in vacuo. Following this, distillated water (2.5 mL) was added so as to hydrate the thin layer film. After brief sonication (2-3 min), the pH of the resulting liposome suspension was adjusted to 4. A solution of 3-ethoxypropion-aldehyde 14 (10.42 mg, 10.21 μmoles) was added and the suspension was sonicated for 10min. The post-coupled liposome was left for 2d at room temperature then freeze-dried overnight. 19 was isolated by chromatography on silica column using methanol/dichloromethane (1:27) then (1:18) to remove the product. ¹H NMR (400 MHz, CDCl₃) and ¹³C NMR (100 MHz, CDCl₃) as above; ESI-MS [M−H +Na]=652.1.

Cholesteryl oxime-PEG₂₀₀₀lipid (21): PEG₂₀₀₀ bis-propionaldehyde™ 20 (27.3 mg, 13.7 μmoles) and Cholesteryl-aminoxy-lipid 25 (14.9 mg, 27.4 μmoles) were dissolved in CDCl₃. The reaction mixture was left overnight to give the oxime 21. ¹H NMR (400 MHz, CDCl₃) 0.68 (s, 6 H, Chol C-1 8), 0.83, 0.82 (2×d, 12 H, J=6.5 and 2.0 Hz), 0.91 (d, 6H, J=6.4, Chol C-21), 1.0 (s, 6 H, Chol C-19), 0.94 -2.10 (Chol C-1, 2, 4, 7, 8, 9, 11, 12, 14, 15, 16, 17, 20, 22, 23, 25), 2.32 (m, 4H, Chol C-24), 2.5 and 2.69 (2q, J=5.6, 4H, CH₂CH═N), 3.31 (m, 4H, O(CO)NHCH₂CH₂), 3.52 (m, 4H, J=5.6, CH₂O), 3.64 (m, 196H, OCH₂CH₂), 3.81 (m, 4H, CH₂), 4.4 (m, 4H, Chol C-3), 4.5 and 4.6 (2s, 2H, (CO)CH₂ONH), 4.8 (m, 2 H, Chol-O(CO)NH), 5.35 (m, 2H, Chol C6), 6.7 (s br, 1H, NH), 6.8 (s br, 1H, NH), 6.88 and 7.60 (2t, 2H, J=5.6, CH═N). ¹³C NMR (100 MHz, CDCl₃) 11.8 (C-18). 18.7 (C-21), 19.3 (C-19), 21.0 (C-11), 22.5 (C-27), 22.7 (C-26), 23.8 (C-23), 24.3 (C-15), 26.7 (CH₂), 28.0 (C-25), 28.1 (C-2), 28.2 (C-16), 30.1 (CH₂CHN), 31.8 (C-7), 31.9 (C-8), 35.8 (C-20), 36.2 (C-22), 36.6 (C-10), 37.0 (C-1), 38.5 (C-24), 39.5-39.7 ((C-12, C4), O(CO)NHCH₂CH₂ overlapping), 40.7 (C-4), 42.3 (C-13), 50.0 (C-9), 56.1 (C-17), 56.7 (C-14), 61.6 (OCH₂CH₂), 70.0 and 67.5 (CH₂O), 70.22-70.9 (OCH₂), 72.5 (C-3), 72.8 ((CO)CH₂ONH₂), 122.4 (C-6),139.8 (C-5), 151.4 and 151.7 (CH═NO), 158.3 (OCONH), 171.6 (NH(CO)CH₂ONH₂),

Post-Coupling Reaction

Cholesteryl oxime-PEG₂₀₀₀lipid (21): DMPC (15.51 mg, 22.28 μmoles) and Cholesteryl-aminoxy-lipid 25 (14.9 mg, 27.4 μmoles) were combined in chloroform to prepare liposomes of DMPC/25 (45:55, w:w).The solution was transferred into a round bottom flask and organic solvent were removed under reduced pressure giving a thin lipid film that was dried in vacuo. Following this, distillated water (6 mL) was added so as to hydrate the thin layer film. After brief sonication (2-3 min), the pH of the resulting liposome suspension was adjusted to 4. A solution of PEG₂₀₀₀ bis-propionaldehyde™ 20 (27.3 mg, 13.7 μmoles) was added and the suspension was sonicated for 10 min. The post-coupled liposome was left for 2d at room temperature then freeze-dried overnight. 21 was isolated by chromatography on silica column using methanol/dichloromethane (1:19) then (1:9) to remove the product. ¹H NMR (400 MHz, CDCl₃) and ¹³C NMR (100 MHz, CDCl₃) as above.

Cholesteryl amine (23): Cholesteryl chloroformate 22 (7.5 g, 0.0167 mol) was dissolved in ethylene-1,2-diamine (180 ml) and the mixture stirred for 18 h. The reaction was quenched with water and extracted with dichloromethane. The organic extracts were dried (MgSO₄) and the solvent removed in vacuo to afford a residue which was purified by flash column chromatography [CH₂Cl₂:MeOH: NH₃ 192:7:1→CH₂Cl₂:MeOH:NH₃ 92:7:1 (v/v)] giving the pure product 23 (5.5, g, 0.0116, 73%) as a white solid (mp 175-177° C.): FTIR (nujol mull) νₘₐₓ 3338 (amine), 2977 (alkane), 2830 (alkane), 1692 (carbamate) cm⁻¹; ¹H NMR (CDCl₃) δ 0.66 (3 H, s, H-18), 0.838-0.854 (3 H, d, H-27 (J=6.4 Hz)), 0.842-0.858 (3 H, d, H-26 (J=6.4 Hz)), 0.890-0.906 (3 H, d, H-21 (J=6.4 Hz)), 0.922 (3 H, s, H-19), 1.02-1.63 (21 H, m, H-1, H-9, H-11, H-12, H-14, H-15, H-16, H-17, H-20, H-22, H-23, H-24, H-25), 1.76-2.1 (5 H, m, H-2, H-7, H-8), 2.22-2 36 (2 H, m, H-4), 2.79-2.81 (2 H, m, H₂NCH₂), 3.197-3.210 (2 H, m, H₂NCH₂CH2), 4.52 (1 H, m, H-3), 5.31 (1 H, s, H-6); ¹³C NMR (CDCl₃) δ 11.78 (C-18), 18.64 (C-21), 19.26 (C-19), 20.96 (C-11), 22.49 (C-26), 22.75 (C-27), 23.7 (C-23), 24.20 (C-15), 27.92 (C-25), 28.09 (C-2), 28.16 (C-16), 31.77 (C-8), 31.81 (C-7), 35.72 (C-20), 36.09 (C-22) 36.46 (C-10), 36.91 (C-1), 38.50 (C-24), 39.43 (C-4), 39.64 (C-12), 42.2 (C-13), 41.70 (H₂NCH₂CH₂), 43.55 (H₂NCH₂), 49.91 (C-9), 56.04 (C-17), 56.59 (C-14), 74.20 (C-3), 122.39 (C-6), 156.39 (C═O); MS (ESI+ve) 473 (M+H); HRMS (FAB+ve) calcd. for C₃₀H₅₃N₂O₂ (M+H) 473.411911 found 473.410704.

Boc-aminoxy cholesteryl lipid (24): Boc-amino-oxyacetic acid 27 (145 mg, 0.758 mmol) in anhydrous dichloromethane was treated successively with DMAP (292 mg, 2.39 mmol), HBTU (373 mg, 0.987 mmol) and amine 23 (272 mg, 0.576 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography (gradient 20% Ethyl acetate/Hexane to 65% Ethyl acetate/Hexane) affording pure Boc-aminoxy cholesteryl lipid 24 (302 mg, 81%). ¹H NMR (400 MHz, CDCl₃) 8.56 (s, 1H, BocNHOCH₂), 8.2 (br, CH₂CONHCH₂), 5.5 (m, 1H, Chol C6), 5.4 (m, 1 H, Chol-O(CO)NH), 4.5 (m, 1H, Chol C-3), 4.3 (s, 2H, (CO)CH₂ONH₂), 3.4 (m, 2H, O(CO)NHCH₂CH₂), 3.3 (m, 2H, O(CO)NHCH₂CH₂), 2.32 (m, 2 H, Chol C-24), 1.46 (s, 3 H, Boc), 0.94-2.10 (Chol C-1, 2, 4, 7, 8, 9, 11, 12, 14, 15, 16, 17, 20, 22, 23, 25), 1.0 (s, 3 H, Chol C-19), 0.89 (d, 3 H, J=6.4, Chol C-21), 0.83, 0.82 (2×d, 6 H, J=6.5 and 2.0 Hz), 0.68 (s, 3 H, Chol C-18); ¹³C NMR (100 MHz, CDCl₃) 169.6 (NH(CO)CH₂ONH₂), 157.9 (Boc), 156.6 (OCONH), 139.7 (C-5), 122.4 (C-6), 82.8 (Boc), 76.2 ((CO)CH₂ONH₂), 74.4 (C-3), 56.6 (C-14), 56.0 (C-1 7), 49.9 (C-9), 42.2 (C-1 3), 40.6 (C4), 39.4-40.6 (C-12, C-4, O(CO)NHCH₂CH₂ overlapping), 38.4 (C-24), 36.9 (C-1), 36.4 (C-10), 36.1 (C-22), 35.7 (C-20), 31.80 (C-8), 321.79 (C-7), 28.1 (C-16 and Boc overlapping), 28.0 (C-2), 27.9 (C-25), 24.2 (C-15), 23.7 (C-23), 22.7 (C-26), 22.5 (C-27), 20.9 (C-11), 19.2 (C-19), 18.6 (C-21) and 11.8 (C-18). ESI-MS 646 [M+H]⁺; HRMS: calculated for C₃₇H₆₄N₃O₆: 646.479512; Found: 646.479874.

Cholesteryl aminoxy lipid (25): Boc-aminoxy cholesteryl lipid 23 (86 mg, 0.067 mmol) in propan-2-ol (3 ml) was then treated with 4M HCl in dioxane (3 ml) and the mixture stirred at room temperature for 3 h. The solvents were removed in vacuo affording aminoxy lipid 25 (37 mg, 98%); ¹H NMR (400 MHz, d₄-MeOD) 5.35 (m, 1H, Chol C6), 4.8 (m, 1 H, Chol-O(CO)NH), 4.5 (s, 2H, (CO)CH₂ONH₂), 4.4 (m, 1H, Chol C-3), 3.3 (m, 2H, O(CO)NHCH₂CH₂), 3.1 (m, 2H, O(CO)NHCH₂CH₂), 2.32 (m, 2 H, Chol C-24), 0.94-2.10 (Chol C-1, 2, 4, 7, 8, 9, 11, 12, 14, 15, 16, 17, 20, 22, 23, 25), 1.0 (s, 3 H, Chol C-19), 0.89 (d, 3 H, J=6.4, Chol C-21), 0.83, 0.82 (2 x d, 6 H, J=6.5 and 2.0 Hz), 0.68 (s, 3 H, Chol C-18); ¹³C NMR (100 MHz, CDCl₃) 171.4 (NH(CO)CH₂ONH₂), 158.3 (OCONH), 140.55 (C-5), 123.2 (C-6), 75.4 ((CO)CH₂ONH₂) 71.9 (C-3), 57.5 (C-14), 57.0 (C-17), 51.0 (C-9), 43.0 (C-13), 40.2 (C-4), 40.0-40.6 (C-12, C4), O(CO)NHCH₂CH₂ overlapping), 39.2 (C-24), 37.8 (C-1), 37.3 (C-10), 36.9 (C-22), 36.6 (C-20), 32.7 (C-8), 32.6 (C-7), 28.9 (C-16), 28.8 (C-2), 28.7 (C-25), 24.9 (C-15), 24.5 (C-23), 23.2 (C-26), 22.9 (C-27), 21.8 (C-1 1), 19.7 (C-19), 19.2 (C-21) and 12.3 (C-18). ESI-MS 546 [M +H]⁺.

DSPE-AmBoc (28): DSPE 26 (150 mg, 0.200 mmol), Boc-amino-oxyacetic acid 27 (43 mg, 0.222 mmol) in anhydrous dichloromethane was treated successively with DMAP (91 mg, 0.732 mmol) and HBTU (93 mg, 0.244 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography (gradient Eluent B 90: CH₂Cl₂ 10 to Eluent B) affording pure DSPE-AmBoc 28 (103 mg, 0.112 mmol; 56%); ¹H NMR (CDCl₃) δ 0.9 (overlapping triplets, 2×CH₃, J=6.6 Hz), 1.2-1.35 (br m, 56 H, Stearyl CH₂), 1.47 (Boc), 1.6 (m, 4H, CH₂CH₂COO), 2.3 (4H, overlapping triplets, CH₂CH₂COO, J=7.7, 7.7), 3.50 (2H, m, OPOCH₂CH₂N), 3.93 (4H, m, Glycerol c CH₂ and OPOCH₂CH₂N), 4.15 and 4.38 (2H, m, Glycerol a CH₂), 4.35 (2H, s, COCH₂ONHBoc), 5.20 (1H, m, Glycerol b CH₂), 8.2 (1H, br s, NH), 9.34 (1H, br s, NH). ¹³C-NMR (CDCl₃) δ 14.08 (ω-1 CH₂ stearic esters), 22.67 (ω-2 CH₂ stearic esters), 24.91 and 24.85 (2×CH₂×CH₂COO), 28.18 (Boc), 29.16 to 29.71 (stearic ester methylenes), 31.91 (ω-3 CH₂ stearic esters), 34.07 and 34.25 (CH₂CHCOO×2), 39.93 (CH₂CH₂NHCO), 62.61 (glycerol-1 CH₂), 63.71 (d, glycerol-3), 64.39 (d, OPOCH₂CH₂N), 70.25 (d, glycerol-2), 75.33 (COCH₂ONHBoc), 82.2 (Boc), 157.75 (Boc), 169.91 (COCH₂ONHBoc), 173.28 and 173.604 (stearic esters CO); ESI-MS 919.70 (M−H)⁺.

DSPE-aminoxy (29): DSPE-AmBoc 28 (50 mg, 0.054 mmol) was dissolved in 2-propanol (1 ml): 4M HCl in dioxane (2 ml) and the mixture stirred at r.t for 1 h. The solvent was removed at 50° C. at reduced pressure and the residue suspended in ether. Sonication of the mixture followed by centrifugation (process repeated 3×) afforded the product 29 as a white solid (19 mg, 0.022 mmol, 41%). ¹H NMR (CDCl₃: MeOD; 2:1 v/v) δ 0.84 (overlapping triplets, 2×CH₃, J=6.7 Hz), 1.2 -1.35 (br m, 56 H, Stearyl CH₂), 1.6 (m, 4H, CH₂CH₂COO), 2.3 (4H, overlapping triplets, CH₂CH₂COO, J=7.7 , 7.7), 3.47 (2H, m, OPOCH₂CH₂N), 3.93 (4H, m, Glycerol c CH₂ and OPOCH₂CH₂N), 4.15 and 4.38 (2H, m, Glycerol a CH₂), 4.50 (2H, s, COCH₂ONH₂), 5.20 (1H, m, Glycerol b CH₂). ¹³C-NMR (CDCl₃: MeOD; 2:1 v/v) δ 14.37 (ω-1 CH₂ stearic esters), 23.26 (ω-2 CH₂ stearic esters), 25.51 and 25.53 (2×CH₂CH₂COO), 29.72 to 30.33 (stearic ester methylenes), 32.55 (ω-3 CH₂ stearic esters), 34.67 and 34.81 (CH₂CH₂COO×2), 40.62. (CH₂CH₂NHCO), 62.92 (glycerol-1 CH₂), 65.06 (d, glycerol-3), 65.43 (d, OPOCH₂CH₂N), 70.79 (d, glycerol-2), 72.68 (COCH₂ONH), 174.19 and 174.59 (stearic esters CO); ESI-MS 819 (M−H)⁺.

Dioctadecylcarbamoylmethyl-carbamic acid tert-butyl ester (32): Boc-glycine 31 (307 mg, 1.75 mmol, leq.) and dioctadecylamine 30 (915 mg, 1.75 mmol, 1 eq.) was dissolved in dry chloroform (30 mL) under anhydrous conditions. HBTU (797.6 mg, 2.10 mmol, 1.2 eq.) and DMAP (642.3 mg, 5.26 mmol, 3 eq.) were added to the solution. The reaction was stirred at room temperature under argon until TLC indicated the reaction had gone to completion (˜12 hrs). Solvents were removed in vacuo. The residue dissolved in CH₂Cl₂ (50 mL) and extracred with water (3×50 mL). The combined aqueous extracts were back extracted with CH₂Cl₂ (3×50 mL) and 2 CHCl₃: 1 MeOH (2×50 mL). The organic extracts were combined, dried (MgSO₄) and concentrated. The resulting yellow oil was purified by silica gel column chromatography [Petroleum→Petroleum:Diethylether 8:2 (v/v)], rendering the title compound 32 (801 mg, 70%) as a yellow oil: FTIR (film) νₘₐₓ 3329 (amide NH), 2989 (alkyl), 2830 (alkyl), 1656 (amide C═O) cm⁻¹, ¹H NMR (CDCl₃) δ 0.78-0.91 (6 H, m, 2×CH₃), 1.1-1.34 (60 H, m, alkylchain CH₂'s), 1.35-1.6 (13 H, m, C(CH₃)₃ and N(CH₂CH₂-alkylchain)₂), 3.01-3.12 (2 H, m, NCH₂), 3.23-3.41 (2 H, m, NCH₂), 3.85-3.96 (2 H, m, NHCH₂CO), 5.50-5.59 (1 H, br, s, amide NH); ¹³C NMR (CDCl₃) δ 14.49 (2×CH₃), 23.07 (N(CH₂CH₂-alkylchain)₂), 27.27-32.31 (30 CH₂, alkylchain), 42.54 (NHCH₂CO), 46.47 (N(CH₂CH₂-alkylchain)₂), 47.28 (N(CH₂CH₂-alkylchain)₂), 79.76 (C(CH₃)₃), 156.19 (C(CH₃)₃COCO), 167.94 (CON-alkylchains); MS (ESI +ve) 679 (M+H).

2-Amino-N,N-dioctadecyl-acetamide (33): Boc-Amine 32 (260 mg, 0.38 mmol, 1 eq.) was dissolved in dry CH₂Cl₂ (5 mL) under anhydrous conditions. Triflouroacetic acid (3 mL) was added to the solution cautiously. The reaction was stirred at room temperature under a positive flow of nitrogen until TLC indicated the reaction had gone to completion (˜2 hrs). The solvents were removed in vacuo rendering the desired compound 33 (200 mg, 92%) as an off-white solid: FTIR (nujol mull) νₘₐₓ 3130 (amine), 2825 (CH₂), 1678 (amide) cm⁻¹; ¹H NMR (CDCl₃) δ 0.85-0.92 (6 H, t, 2×CH₃), 1.17-1.43 (60 H, m, 30 CH₂'s alkylchain), 1.45-1.60 (4 H, m, N(CH₂CH₂-alkylchain)₂), 3.01-3.11 (2 H, m, NCH₂CH₂-alkylchain), 3.21-3.3 (2 H, m, N(CH₂CH₂-alkylchain), 3.85-3.95 (2 H, m, CH₂NH₂); ¹³C NMR (CDCl₃) δ 14.46 (2×CH₃), 23.09 (2CH₂CH₃), 27.14-32.35 (30CH₂, alkylchain), 40.49 (CH₂NH₂), 46.91 (N(CH₂CH₂-alkylchain), 47.59 (N(CH₂CH₂-alkylchain), 165.46 (C═O); MS (FAB +ve) 579 (M⁺); HRMS (FAB +ve) calcd. for C₃₈H₇₉N₂O (M+H) 579.619385, found 579.619241.

2-(Boc-aminooxy)-N-dioctadecylcarbamoylmethyl-acetamide (34): Amine 33 (255 mg, 0.442 mmol) and Boc-amino-oxyacetic acid 27 (89 mg, 0.464 mmol) were dissolved in anhydrous DCM (50 ml) and then treated with DMAP (187 mg, 1.53 mmol) and HBTU (192 mg, 0.510 mmol) and stirred at r.t. for 14 h. The mixture was quenched with 7% aqueous Citric acid and the aqueous phase extracted with DCM. The organic portions were dried (MgSO₄) and concentrated in vacuo. The resultant residue was purified by flash column chromatography (30-40% EtOAc/Hexanes) to afford pure 34 (282 mg, 0.375 mmol, 85%). ¹H NMR (CDCl₃) δ 0.80 (6 H, m, 2×CH₃), 1.1-1.34 (60 H, m, alkylchain CH₂'s), 1.35-1.6 (13 H, m, C(CH₃)₃ and N(CH₂CH₂-alkylchain)₂), 3.0-3.1 (2 H, m, NCH₂), 3.20-3.40 (2 H, m, NCH₂), 3.85-3.96 (2 H, m, NHCH₂CO), 4.30 (2H, s, CH₂ONHBoc), 7.5 and 7.7 (2 H, 2×br, s, amide NH); ¹³C NMR (CDCl₃) δ 14.13 (2×CH₃), 22.66 (N(CH₂CH₂-alkylchain)₂), 26.85-32.31 (30 CH₂, alkylchain), 40.64 (NHCH₂CO), 46.27 (N(CH₂CH₂-alkylchain)₂), 47.01 (N(CH₂CH₂-alkylchain)₂), 75.44 (CH₂ONHBoc), 82.41 (C(CH₃)₃), 156.70 (C(CH₃)₃COCO), 167.02 (CON-alkylchains) and 168.58 (CO amide); MS (ESI+ve) 752 (M+H)⁺ and 774.30 (M+Na)⁺.

2-Aminooxy-N-dioctadecylcarbamoylmethyl-acetamide (35): Boc-aminooxy 34 (80 mg, 0.106 mmol) was dissolved in DCM (1.5 ml) and treated with TFA (1.5 ml). The mixture was stirred at r.t. for 15 min. and then concentrated in vacuo affording a solid residue of pure 35 (81 mg, 100%). ¹H NMR (CDCl₃) δ 0.92 (6 H, m, 2×CH₃), 1.25-1.4 (60 H, m, alkylchain CH₂'s), 1.55-1.7 (4H, m, N(CH₂CH₂-alkylchain)₂), 3.0-3.1 (2 H, m, NCH₂), 3.20-3.40 (2 H, m, NCH₂), 4.15 (2 H, m, NHCH₂CO), 4.60 (2H, s, CH₂ONH₂), 8.0 (1 H, br, s, amide NH); ¹³C NMR (CDCl₃) δ 14.1 (2×CH₃), 22.6 (N(CH2CH₂-alkylchain)₂), 26.88-31.95 (30 CH₂, alkylchain), 40.6 (NHCH₂CO), 46.2 (N(CH₂CH₂-alkylchain)₂), 47.0 (N(CH₂CH₂-alkylchain)₂), 72.2 (CH₂ONHBoc), 167.6 (CON-alkylchains) and 168.6 (CO amide); HRMS: Calculated for C₄₀H₈₂N₃O₃=652.635619. Found 652.636215.

Boc-aminoxy-(dPEG₄)₂-CO₂H (38): The Boc-aminoxy-dPEG₄)₂-CO₂H 38 was synthesised using a standard peptide solid phase synthesis strategy: Chlorotrityl Chloride resin (1.27 mmol/g loading, 55 mg, 0.070 mmol) was swollen in DCM for 16 h. The first acid was loaded onto resin by treating the resin with N-Fmoc-amido-dPEG₄™-acid (102 mg, 0.209 mmol) and Hunig base (60 μl, 0.349 mmol) in DMF (15 ml) for 1 hour. Fmoc deblocking was achieved by using piperidine (20%) in DMF (2×5 mins) followed by extensive washing with DMF. Next the resultant resin-bound free amine 36 was reacted with N-Fmoc-amido-dPEG₄™-acid (102 mg, 0.209 mmol), activated with HBTU (132.5 mg, 0.209 mmol) in Hunig base (60 μl, 0.349 mmol) in DMF (15 ml) for 1 hour. (For each coupling step, 3 equivalent of amino acid, 5 equivalents of DIEA and 3 equivalents of HBTU were used. Each coupling was carried out for 1 hour followed by capping with acetic anhydride (10%) in DMF in the presence of 3 equivalents of DIEA.) Finally, Boc-amino-oxyacetic acid 27 (40 mg) was coupled to yield the resin bound product 37. The compound was cleaved using 3 mL of a solution consisting of 50% trifluoroethanol in DCM over 4 hours to yield a crude residue 38 (40 mg, 0.058 mmol). δ_{H} (CDCl₃) 1.48 (9H, Boc), 2.51 (2H, t, J=6.1 Hz, ˜CH2CO2H), 2.59 (2H, t, J=6.05, ˜CH2CONHCH2˜), 3.45 and 3.52 (2H and 2H, m, CONHCH2CH2), 3.55-3.7 (28H, m, CH2OCH2 and CH2OCH2), 3.77 (4H, m, NHCH2CH2O), 4.34 (2H, s, BocHNOCH2CONH), 7.0 (1H, m, BocNHO), 7.9 (1 H, m, CH2NHCOCH2) and 8.3 (1H, m, CH2NHCOCH2). δ_{C} (CDCl₃) 28.2 (Boc), 35.1 (˜CH2CO2H), 36.8 (˜CH2CONHCH2˜), 38.98 and 39.24 (CONHCH2CH2), 66.7 and 67.3 (CH2CH2CO), 69.6 and 69.9 (NHCH2CH2O), 70.3-70.7 (CH2OCH2 and CH2OCH2), 75.8 (BocHNOCH2CONH), 82.5 (quaternary, Boc), 158 (CO, Boc), 169.3 and 171.8 (quaternary, CH2NHCOCH2) and 173.6 (quaternary, CO2H). ESI-MS 684.30 (M−H)⁺.

Boc-aminoxy-dPEG₄)₂-cholesteryl lipid (39) (BocCPA): Boc-aminoxy-dPEG₄)₂-CO₂H 38 (40 mg, 0.058 mmol) in anhydrous dichloromethane was treated successively with DMAP (22 mg, 0.18 mmol), HBTU (24 mg, 0.063 mmol) and cholesteryl amine 23 (28 mg, 0.0.06 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography (gradient DCM:MeOH:H₂O) affording pure Boc-aminoxy-dPEG₄)₂-cholesteryl lipid 39 (47 mg, 0.0411 mmol, 71%). ¹H NMR (400 MHz, CDCl₃:MeOD) 5.32 (m, 1H, Chol C6), 4.35 (m, 1H, Chol C-3), 4.28 (s, 2H, (CO)CH₂ONH₂), 3.67 (4H, m, NHCH2CH2O), 3.56-3.61 (24H, m, CH2OCH2 and CH2OCH2), 3.56 (2H, m, CH2CH2CO), 3.50 (2H, m, CH2CH2CO), 3.35 and 3.43 (2H and 2H, m, CONHCH2CH2), 3.24 (m, 2H, CholO(CO)NHCH₂CH₂), 3.18 (m, 2H, CholO(CO)NHCH₂CH₂), 2.42 (4H, m, ˜CH2CO2H and ˜CH2CONHCH2˜), 2.27 (m, 2 H, Chol C-24), 1.46 (s, 3 H, Boc), 0.94 -2.10 (Chol C-1, 2, 4, 7, 8, 9, 11, 12, 14, 15, 16, 17, 20, 22, 23, 25), 1.0 (s, 3 H, Chol C-19), 0.89 (d, 3 H, J=6.4, Chol C-21), 0.83, 0.82 (2×d, 6 H, J=6.5 and 2.0 Hz), 0.68 (s, 3 H, Chol C-18); ¹³C NMR (100 MHz, CDCl₃) 173.6 (quaternary, CO2H), 173.3, 172.8 and 170.5 (NH(CO)CH₂ONH₂), 158.5 (Boc), 156.6 (OCONH), 140.166 (C-5), 122.92 (C-6), 82.61 (Boc), 75.77 ((CO)CH₂ONH₂), 74.99 (C-3), 70.4-70.8 (CH2OCH2 and CH2OCH2), 69.81 and 70.04 (NHCH2CH2O), 67.56 and 67.53 (CH2CH2CO), 56.7 (C-14), 56.55 (C-17), 50.5 (C-9), 42.7 (C-13), 40.63 and 39.81 (CholO(CO)NHCH₂CH₂) 40.14 (C-4), 39.88 and 39.58 (CONHCH2CH2), 39.25 (C-12), 38.94 (C-24), 37.3 (C-1), 36.9 (C-10), 36.95 (˜CH2CONHCH2˜), 36.90 (˜CH2CO2H), 36.55 (C-22), 36.17 (C-20), 32.28 (C-8), 32.26 (C-7), 28.5 (C-16 and C-2 overlapping), 28.36 (Boc and C-25), 24.6 (C-15), 24.17 (C-23), 22.99 (C-26), 22.73 (C-27), 21.4 (C-11), 19.6 (C-19),18.96 (C-21) and 12.11 (C-18). ESI-MS 1162.40 [M+K].

Cholesteryl-(dPEG₄)₂-aminoxy lipid (40) (CPA): Boc-aminoxy-dPEG₄)₂-cholesteryl lipid 39 (40 mg, 0.035 mmol) in propan-2-ol (2 ml) was then treated with 4M HCl in dioxane (2 ml) and the mixture stirred at room temperature for 3 h. The solvents were removed in vacuo affording CPA lipid 40 (37 mg, 98%); ¹H NMR (400 MHz, d₄-MeOD) 5.31 (m, 1 H, Chol C6), 4.57 (s, 2H, (CO)CH2ONH₂), 4.38 (m, 1H, Chol C-3), 3.69 (4H, m, NHCH2CH2O), 3.53-3.62 (28H, m, CH2OCH2 and CH2OCH2), 3.37 and 3.43 (2H and 2H, m, CONHCH2CH2), 3.26 (m, 2H, CholO(CO)NHCH₂CH₂), 3.19 (m, 2H, CholO(CO)NHCH₂CH₂), 2.45 (4H, m, ˜CH2CO2H and ˜CH2CONHCH2˜), 2.27 (m, 2 H, Chol C-24), 0.94-1.99 (Chol C-1, 2, 4, 7, 8, 9, 11, 12, 14, 15, 16, 17, 20, 22, 23, 25), 0.97 (s, 3 H, Chol C-1 9), 0.87 (d, 3 H, J=6.4, Chol C-21), 0.80, 0.82 (2×d, 6 H, J=6.5 and 2.0 Hz), 0.64 (s, 3 H, Chol C-18); ¹³C NMR (100 MHz, CDCl₃) 173.6 (quaternary, CO2H), 173.3, 172.8 and 170.5 (NH(CO)CH₂ONH₂), 157.6 (OCONH), 140.16 (C-5), 122.94 (C-6), 75.03 (C-3), 71.90 ((CO)CH₂ONH₂), 70.4-70.83 (CH2OCH2 and CH2OCH2), 69.54 and 70.14 (NHCH2CH2O), 67.62 (2×CH2CH2CO overlapping), 57.12 (C-14), 56.56 (C-17), 50.50 (C-9), 42.7 (C-13), 40.54 and 39.91 (CholO(CO)NHCH₂CH₂) 40.14 (C-4), 39.88 (C-12), 39.38 and 39.65 (CONHCH2CH2), 38.94 (C-24), 37.3 (C-1), 36.95 (C-10), 36.87 (˜CH2CONHCH2˜), 36.78 (˜CH2CO2H), 36.55 (C-22), 36.17 (C-20), 32.28 (C-8), 32.26 (C-7), 28.5 (C-16 and C-2 overlapping), 28.36 (C-25), 24.6 (C-15), 24.17 (C-23), 22.98 (C-26), 22.73 (C-27), 21.42 (C-11), 19.6 (C-19), 18.96 (C-21) and 12.11 (C-18). ESI-MS 1102.50 [M+K+Na]⁺. Analytical HPLC: 1 peak, RT 31 min.

Biological and Biophysical Evaluation:

General: Dioleoylphosphatidyl-ethanolamine (DOPE), N-(lissamine rhodamine B sulfonyl)-phosphatidylethanolamine (Rh-DSPE), distearoyl-phosphatidylcholine (DSPC), PEG-2000 distearoylphosphatidylinethanolamine (DSPE-PEG2000) were purchased from Avanti Lipid (Alabaster, Ala., USA). Bis-aldehyde polyethylene glycol (PEG²⁰⁰⁰(CHO)₂) and bis-aldehyde polyethylene glycol (PEG³⁵⁰⁰(CHO)₂) were purchased from (Nektar, Huntsville, Ala., USA). Plasmid pCMVβ was produced by Bayou Biolabs (Harahan, La., USA). DC-Chol was synthesised in our Laboratory^{[27]}. Mu-peptide was synthesised by M. Keller by standard Fmoc based Merrifield solid phase peptide chemistry on Wang resine^{[43]}. The cationic lipid CDAN was synthesized according to Keller and al, Biochemistry 2003, 42, 6067. Cholesterol, carbohydrates and all other chemicals were purchased from Sigma Aldrich (Dorset, UK) unless otherwise stated. All other chemicals were reagent grade.

Preparation of Liposomes: DC-Chol (7.5 mg, 15 μmol) and DOPE (7.5 mg, 10 μmol) were combined in dichloromethane. The solution was transferred to a round-bottomed flask (typically 50 ml) and organic solvent removed under reduced pressure (rotary evaporator) giving a thin-lipid film that was dried for 2-3 h in vacuo. Following this, 4 mM HEPES buffer, pH 7.2 (3 ml) was added to the round-bottomed flask so as to hydrate the thin-lipid film. After brief sonication (2-3 min.) under argon, the resulting cationic liposome suspension (lipid concentration of 5 mg/ml) was extruded by means of an extruder device (Northern lipid). Initially, three times through two stacked polycarbonate filters (0.2 μm) and then ten times through two stacked polycarbonate filters (0.1 μm) to form small unilamellar cationic liposomes (average diameter 105 nm according to PCS analysis). Lipid concentrations (approx. 4-4.8 mg/ml) were determined by Stewart assay.

Preparation of Liposome:Mu:DNA (LMD) and Liposome:DNA (LD) complexes: Initially, mu:DNA (MD) particles were prepared by mixing as follows. Plasmid DNA stock solutions (typically 1.2 mg/ml) were added to a vortex-mixed, dilute solution of mu peptide (1 mg/ml) in 4 mM HEPES buffer, pH 7.2. The final mu:DNA ratio was 0.6:1 w/w, unless otherwise stated, and final plasmid DNA concentration was 0.27 mg/ml. MD containing solutions were then added slowly under vortex conditions to suspensions of extruded cationic liposomes (typically approx. 4.5 mg/ml), prepared as described above, resulting in the formation of small LMD particles with narrow size distribution (120±30 nm) as measured by PCS. Final lipid:mu:DNA ratio 12:0.6:1 w/w/w. A solution of sucrose (100%, w/v) in 4 mM HEPES buffer, pH 7.2, was then added to obtain LMD particle suspensions in 4 mM HEPES buffer, pH 7.2 containing 10% w/v sucrose at the desired DNA concentration (final DNA concentration typically 0.14 mg/ml) and the whole stored at −80° C. Liposome:DNA (LD) complexes (lipoplexes) were prepared for experiments with a lipid:DNA ratio of 12:1 (w/w) following the same protocol without the addition of Mu peptide.

Particle size measurements: The sizes of the lipoplexes were evaluated after 30 min exposure at 37° C. to biological media by Photon Correlation Spectroscopy (N4 plus, Coulter). The chosen DNA particular concentration was compatible with in vitro condition (1 μg/ml of DNA). The parameters used were: 20° C., 0.089 cP, reflexive index of 1.33, angle of 90° C., 632.8 nm. Unimodal analysis was used to evaluate the mean particle size in Optimem. Size distribution program using the CONTIN algorithm was utilised to separate the sub-population of small serum particle of less than 50nm and to extracted the calculated size of lipoplexes in Optimem +10% FCS.

Transfection of HeLa cells: Cells were seeded in a 24-wells culture plate in DMEM supplemented with 10% FCS and grown to approximately 70% confluence for 24 h at 37° C. in the presence of 5% CO₂. The cells were washed in PBS before the transfection media was administered to each well (0.5 ml of solution of 0, 50 or 100% Foetal Calf Serum in Dubelco OptiMem). 5 μl at 100 μg/ml DNA (nls βgal) of LMD were transfected onto Hella Cells for 30 min. Cells were then rinsed 3 times with PBS and incubated for a further 48 h in DMEM supplemented with 10% FCS prior to processing for β-Gal expression by using standard chemiluminescent reporter gene assay kit (Roche Diagnostics, GmbH Cat No. 1 758 241).

Kinetic of coupling of carbohydrate and polymer on liposomes containing cholesterol-based aminoxy-lipids: Liposomes containing DSPC:CholONH₂ 25 (50:50, m:m) or DSPC:Chol:CPA 40 (50:35:15, m:m:m) were formulated by lipid film method followed by hydration in H₂O at 55° C. (5 mg/ml). The resulting suspension was sonicated for 30 minutes to obtain a homogeneous solution of 100 nm vesicles. An excess (5 molar equivalents) of carbohydrate solutions in H₂O were added to the liposomes at pH 5 (37° C.). Alternatively for PEG coupling equimolar quantities of PEG²⁰⁰⁰(CHO)₂ were incubated with the aminoxy-lipid containing liposomes at pH 7. At different time points, aliquots were analysed by HPLC. The reactivity of the aminoxy-lipid was followed using cholesterol or DSPC as an internal standard. New peaks were isolated and characterized using mass spectrometry.

In vivo functionality of a liposomes containing a cholesterol-based aminoxy-lipid modified by lactose or PEG²⁰⁰⁰(CHO)₂: DSPC/CHOUCPA 40/DSPE-Rhd (50:35:15:1) liposomes were incubated with a defined percent of PEG²⁰⁰⁰(CHO)₂ for 3 days or an excess of lactose. 200 μl of the resulting liposome solutions (2 mg/ml) were injected intravenously into the tail vain of a balb-c mouse (triplicate). After 30 minutes, the animals were sacrificed and the organ repartition was assessed. The fluorescently Rhodamine-labeled lipid, DSPE-Rhd was extracted from the organs by solvent extraction and used for quantification by fluorescense spectroscopy. Results were represented as percent of the total detected dose.

Coupling of carbohydrate and PEG onto liposomes containing a non-cholesterol-based aminoxy-lipids 29 and 35: Liposomes containing DSPC:lipid 35 (50:50, m:m) or DSPC:DOTAP:DSPE-ONH₂ 29 (25:25:50, m:m) were formulated by the, lipid film method followed by hydration in H₂O at 55° C. (5 mg/ml). The resulting suspension was sonicated for 30 minutes to obtain a homogeneous solution unilamellar vesicles. An excess of galactose solution (5 molar equivalent) was added to the liposomes at (pH 5, 37° C., 24 hours). An excess of PEG³⁵⁰⁰(CHO)₂ was incubated with the liposomes at pH 7 for 24 hours. Aliquots were analysed by HPLC. New peaks were isolated and identified using mass spectrometry.

Coupling of a protein (transferrin) onto liposomes containing an aminoxy-lipid: 1 ml of liposome DSPC/Chol/CPA 40/Rhodamine-DSPE (49.5/451510.5) was prepared at 5 mg/ml in HEPES 20mM pH 6.8 using the lipid-film method. 22.2 μl of a 10 mg/ml holo-transferrin (Tf) solution in 20 mM HEPES, 150 mM NaCl, pH=6.8 was oxidized with 0, 0.56 and 5.56 μl of sodium periodate solution at 50 mM in H₂O at pH=6.5. The resulting mix was left to incubate overnight at 4° C. prior to quenching with 2 μl of ethylene glycol. The resulting proteins (not oxidized, oxidized by 10 or 100 equivalent of NaIO₄) were then dialysed extensively (24 h, 4 media change with a 10 000 Mw membrane in a mini-slide a lyser) against 20 mM HEPES, 150 mM NaCl, pH=6.8. The volume of the final transferrin mix (32.5 ul) was adjusted and added to 150 μl of liposome and 37.5 ml of the saline HEPES buffer. This corresponds to 10 equivalent of Tf per mol of aminoxy-lipid. The mix was left to incubate overnight at 4° C. Half of the resulting liposome-Tf was submitted to an inverted-sucrose gradient separation. The fraction of the sucrose gradient were collected and analysed by HPLC and gel electrophoresis to determine the presence of the liposome and/or the excess Tf. The red-band of liposome due to the rhodamine-lipid was easily separated in the top-fraction (10% sucrose). Un-bound Tf remained in the bottom fraction (65% sucrose). Excess sucrose of the liposome fraction was then removed by dialysis against 20 mM HEPES, 150 mM NaCl, pH=7.4.

Coupling of an antibody onto cationic liposomes containing an aminoxy-lipid (CPA): 2 mg of Rabbit IgG (Sigma) was dissolved in 1 mL NaOAc (20 mM), NaCl (0.15M) pH 5.9. In a separate tube, 1 mL of fresh H₅IO₆ was prepared and the two tubes combined and left at room temperature for 1.5 h. The reaction was quenched by the addition of 0.5 mL ethylene glycol prior to transferring the whole reaction mixture into a dialysis tube (Spectrum Labs, USA; MWCO 12000-14000) and dialyzed against 0.1M K₂HPO₄/0.1% TritonX for 16 h. The solution was recovered and the IgG^{ox} concentration determined by the BCA assay. 80 μL CDAN/CPA/DOPE (20:30:50, m/m/m; 2 mg/mL) and 100 μL oxidized IgG^{ox} (0.94 mg/mL) were incubated at 37° C./16 h and 90 μL injected into the HPLC for analysis.

Results and Discussion:

Synthesis of Neoglycolipids: Each member of the targeted family of neoglycolipids consisted of a cholesterol bearing lipid and an oligosaccharide molecule bound together via a linker. The whole synthetic approach was divided in two parts; firstly the synthesis of a lipid containing the linker and secondly the chemioselective coupling of this lipid with chosen sugar molecules. The key to this strategy is the formation of a hydroxylamine (FIG. 1).

This synthesis of the Boc-protected lipid 8 was originally designed based on a convergent methodology using readily available aminoalcohols as starting materials with a complementary blocking group strategy for the amine group. This previously published methodology allowed the preparation of this polyamide-based lipid for gene transfer with little modification^{[27]}.

As mentioned, the glycosylation of hydroxylamino derivatives offers an elegant solution to our synthetic requirements. The commercially available O-(Carboxymethyl)hydroxyl-amine hydrochloride was first Boc-protected ano then reacted with N-hydroxysuccinimide and N,N′-dicyclohexylcarbodiimide (DCC) resulting in the corresponding activated ester. This compound was treated immediately in situ with lipid 8 in THF at pH 8, affording a protected hydroxylamine. After a very straightforward deprotection with aqueous trifluoroacetic acid, the synthesis of the hydroxylamino lipid 11 was completed.

At this stage, we investigated the potential of our chemoselective coupling by reacting the lipid 11 with a number of commercially available non-protected oligosaccharides. This reaction was conducted under mild conditions using a solvent system of DMF and aqueous acetic acid pH 4 Buffer (1:1) which facilitates solubility of both sugar and lipid. As shown in FIG. 2 the reactants are in dynamic equilibrium with the open chair protonated intermediate. In order to force the equilibrium to product formation, an excess of sugar was added. Due to the amphiphilic nature of the neoglycolipid product, isolation during workup was found to be difficult as a result of micelle and foam formation. Solubility problems also hampered the isolation, purification and analytical process. Reaction times and yields varied depending on the carbohydrate used (Table 1).

TABLE 1
Yields, reaction times and diastereoselectivity
of glycosylation of product 11.

| Product | Sugar | Times (days) | Yield (%) | β/α |
|---|---|---|---|---|
| 12a | Mannose | 3 | 65 | 82/18 |
| 12b | Glucose | 1 | 80 | 89/11 |
| 12c | Galactose | 1 | 70 | 85/15 |
| 12d | Glucuronic acid | 1 | 60 | 85/15 |
| 12e | Lactose | 4 | 50 | 85/15 |
| 12f | Maltose | 1 | 65 | 87/13 |
| 12g | Maltotriose | 5 | 85 | 85/15 |
| 12h | Maltotetraose | 5 | 40 | 85/15 |
| 12i | Maltoheptaose | 7 | 55 | 85/15 |

Neoglycolid Conformation: Carbohydrate conformations can be ascertained by NMR in solution. The most useful data for conformation at the anomeric centre (C1a) is probably ¹J¹³C1a-H1a. The absolute value of this coupling constant depends upon the orientation of the carbon-hydrogen bond relative to the lone pairs of the ring oxygen, the electronegativity of the substituent at C1 and the nature of electronegative substituents attached to the rest of the molecule. The difference of ¹J¹³C1-H1 between α and β anomer of pyranoses can be used to determine the anomeric configuration. It is firmly established that ¹J(C1-H1eq)>¹J(C1-H1ax) with an approximate difference of 10 Hz. ¹J(C1-H1eq) is usually around 170 Hz and ¹J(C1-H1ax) approximately 160 Hz. Higher values are observed when O-1 is exchanged with more electronegative element as chlorine or fluorine but a 10 Hz difference is usually observed. Carbon-hydrogen coupling constants of furanosides have been investigated and ¹J(C1-H1eq)>¹J(C1-H1ax) but the difference is much smaller (1-3 Hz).

The characterization will be discussed based on the mannose example but the same analysis procedure was used for the other saccharides when NMR analysis conditions were favourable. Four distinct ring structures can be envisaged (FIG. 3). The pyranose forms can be reasonably expected to be favoured over the furanose rings for steric reasons. So out of the two observed compounds in NMR, the main one is probably a pyranose. The secondary observed compound could not be attributed to mutarotation equilibrium because phase sensitive NOESY did not show a cross peak between the two C1a signals (proving it is a distinctive molecule). Therefore, this compound was not attributed to a furanose form because no shift of ¹³C5a was observed and ¹³C1a was not deshielded as has been demonstrated for related substituted furanose equivalents.

We measured ¹J¹³C1a-H1a=167 Hz for the main compound and ¹J¹³C1a-H1a=177 Hz for the secondary one. The absolute value of those ¹J¹³C1-H1 is 10 Hz higher than expected for classical ⁴C₁ conformation but this is explained by the extreme electronegativity of the O-substituted hydroxylamine group that could slightly deform the chair structure. For pyranose rings it has been established that [¹J(C1-H1eq)−¹J(C1-H1ax)]≈10 Hz, therefore it can be easily concluded that the main compound is the β anomer (H1ax) and the secondary compound is the α anomer (H1eq).

¹H phase sensitive NOESY confirmed this conclusion. Nuclear Overhauser effect was observed between H1a and H2a & H3a for the main compound. Considering the above detailed structure, this compound could not be the α pyranosyl anomer because the equatorial H1 cannot interact in space with H3, whereas the β anomer is perfectly able to generate such interactions. No nuclear overhauser effect was observed for H1a of the secondary compound but this could be due to a lack of sensitivity. Hence, in accordance with data from ¹J¹³C1-H1 and NOESY analyses, we concluded that two mannose pyranose a/p forms (20/80) were produced.

The very similar anomeric (β/α) isomers ratio obtained for the neoglycolipids is not surprising (Table 1), all the sugars having an equatorial hydroxyl in C2 but mannose. The ratio obtained for this last compound is surprising because the β anomer is usually reported as sterically less favourable than the α one. A possible explanation is that this reaction could be driven by some secondary interactions (Hydrogen bonding) between the sugar and the hydroxylamine linker, stabilizing the β anomer (this is consistent with the observation that the NMR signal of the β anomer is always much more deshielded than the α one). This anomeric mixture of synthesized glycolipids are not expected to affect greatly the researched biological properties of the lippsomal constructs, therefore we did not attempt the tedious separation of those diasteroisomers by preparative high pressure liquid chromatography.

Biological application:The glyco-modification of LMD was based on the natural ability of miscellar suspension to incorporate into lipid membranes. Firstly LMD were formulated following standard protocol and secondly a suspension of synthesized neoglycolipids miscelles in Hepes Buffer 4 mM pH 7 was added to the LMD and incubated for 30 min at room temperature before usual −80° C. storage. Different percents of all the neoglycolipids produced were tested for stabilization effect but only the longer chain (maltotetraose 12 h and maltoheptaose 12i) exhibited significant properties at less than 10% (data not shown).

The stabilisation effect of neoglycolipid modified LMD was demonstrated by incorporation of 7.5 molar % of compound 12 h or 12i. Lipid layers of liposomes based formulation are known to aggregate after salt or serum exposure. This phenomenon can be followed by measuring the average particle size increase after a fixed time; any stabilization of the LMD particle should be reflected in a reduction of this parameter. It was chosen to measure the size of the lipoplexes by Photon Correlation Spectroscopy (PCS) after 30 min exposure at 37° C. to OptiMem or OptiMem+10% FCS to mimic standard in vitro conditions. It was not possible to analyse the effect with PCS at higher serum percentages, the conditions being too extreme to allow for the taking of meaningful measurements. FIG. 4 describes the percentage of size increase of those lipoplexes.

The results indicate a clear stabilisation of the particle between LMD and standard liposome formulation. Neoglycolipids introduction at 7.5% proved significantly beneficial in OptiMem and 10% serum. 12i incorporation proved to be the most efficient. This result indicates the need of long carbohydrate chains to create efficient molecular brushes on top of those cationic lipid layers.

Even if some degree of stabilization is demonstrated, usually it results in a reduction of the affinity of the positively charged LMD for the negatively charged cell membrane, inducing a drop in the transfection ability of the construct. However in this case, the invitro transfection results indicated an enhancement of the transfection efficiency due to neoglycolipid modification in both 0% and 50% Serum condition (FIG. 5). This result was attributed to a short range protective effect due to these neoglycolipids hindering short range van der waals based interactions between lipid bilayers of similar polarities but not affecting the longer range charge interactions between oppositely charged membranes. The aggregation induced by serum being based primarily on interaction of LMD with negatively charged proteins, the beneficial effect of our neoglycolipids was also lowered with an increasing percentage of serum (no significant benefit in 100% serum).

Synthesis Of Oxime Compounds: Oxime compounds formed from the reaction of simple aldehyde compounds with aminoxy compounds can be characterized by analysis of spectral data, in particular, using NMR spectroscopy. For example, the characteristic chemical shift values in ¹H NMR spectra for the proton carried by the carbon atom (derived from an aldehyde) of an oxime bond is around 6.8 to 7.9 ppm. In ¹³C NMR, the characteristic chemical shift value for the same carbon in, for example, butanal oxime is 154.2 ppm (see Presch Clerc, Tables of Spectral data for Structure Determination of Organic Compounds, 2^{nd} Edition, 1989, Springer-Verlag). These values may be considered as reference values for the study of each condensation product. Furthermore, the conversion of an aldehyde group into oxime bond can be followed by monitoring the disappearance of the peaks that are characteristic of the starting aldehyde. Thus, the chemical shift in ¹H NMR of an aldehyde proton is typically 9.0 to 10.1 ppm and, for example, for C₃H₇—CHO the chemical shift of the aldehyde carbon in the ¹³C NMR is 201.6 ppm.

As shown in FIG. 7, 3-ethoxypropionaldehyde 13 was converted to the 3-ethoxy-propanaldehyde 14 and then reacted with O-(carboxymethyl)-hydroxylamine 15 to give product 16. The reaction took place in a chloroform/DMSO (3/1) mixture using a 1/1 stoichiometry of reactants. As detailed above, the ¹H NMR spectra of product 16 contains two triplets at 6.71 and 7.44 ppm corresponding to the two stereoisomers, cis and trans, of the oxime. In the ¹³C NMR, this is confirmed by the presence of two distinct signals near 150 ppm corresponding to the carbon of the oxime bond for the two isomers. The reaction product showed no significant traces of the aldehyde, as confirmed by the absence of a signal at between 9.0 to 10.1 ppm in ¹H NMR, and around 201 ppm in the ¹³C NMR, that are characteristic of the starting aldehyde.

Two-dimensional COSY (COrrelation SpectroscopY) experiments allow the connectivity of a molecule to be determined by indicating which protons are spin-spin coupled. COSY spectroscopy, when practised with the aid of magnetic field gradients, is a quick method of establishing connectivity. Thus, using ¹H NMR correlated spectroscopy analysis (¹H-¹H COSY NMR experiment), it has been possible to allocate precisely each proton to its neighbours and confirm the formation of this particular bond (FIG. 8).

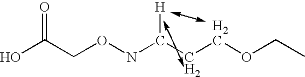

The reaction has also been monitored by mass spectrometry and starting from the aldehyde: ESI MS [M+Na]=124.9, we observed the formation of a compound with ESI MS [M+H]=175.8 and [M+Na]=197.8 in agreement with the molecular weight of the oxime (MW=175.1) (FIGS. 9 and 10).

This experiment has clearly demonstrated the formation of an oxime bond in solution (chloroform) from the reaction between the 2-ethoxy-propanaldehyde and the O-(carboxymethyl)-hydroxylamine.

As shown in FIG. 11, this process was also applied to an aminoxy lipid compound. Thus, Boc-aminoxy-cholesteryl-lipid 17 was deprotected to give the cholesteryl-aminoxy lipid 18 which was reacted with aldehyde 14. The ¹H NMR analysis shows the appearance in the higher fields of two triplets (at 6.88 & 7.61 ppm) corresponding to the two stereoisomers (cis and trans) of the oxime 19. This is also confirmed in ¹³C NMR by the presence of two distinct signals near 150 ppm (at 151.43 ; 151.70 ppm) corresponding to the carbon of the oxime bond for the two isomers. Again the peaks characteristic of the starting aldehyde were no longer present in the NMR of the product.

The formation of this oxime bond was confirmed by a ¹H NMR correlated spectroscopy (¹H-¹H COSY NMR experiment) study (FIG. 12). In addition mass spectrometry showed a value ESI MS [M+Na]=652 in agreement with the molecular weight of the oxime (MW=629.9) (FIG. 13).

HPLC analysis (carried out on a Vydac C4 peptide column) was used to follow the course of the reaction. As can be seen from the HPLC chromatograms, the starting materials give peaks with a retention time of 25.15 min (cholesteryl-aminoxy-lipid 18, see FIG. 14), and 14.60 min (aldehyde 14, see FIG. 15). In comparison, the HPLC chromatograms for the condensation product 19 shows the total disappearance of the peak corresponding to 18, and the appearance of a new peak with a retention time of 29.72 min corresponding to 19 (see FIG. 16).

This oxime forming process was also carried out as a so-called “post-coupling reaction” on the surface of a liposome. That is, the aminoxy-lipid was incorporated in the bilayer of a liposome prior to being reacted with an aldehyde.

A solution of 2-ethoxy propionaldehyde 14 in water was added to a liposome constituted of 55% DMPC and 45% of cholesteryl aminoxy lipid 18 in water at pH 4. After incubation at room temperature overnight, the liposome was lypholised and the different constituents were isolated. TLC analysis showed a peak corresponding to the cholesteryl oxime lipid 19 reference peak; this product was isolated and fully characterized. The ¹H NMR, ¹³C NMR and ¹H-¹H COSY NMR and mass spectra showed that the product is the same cholesteryl oxime lipid 19 as obtained from the reaction carried out in solution.

Similarly, the formation of the oxime 21 (see FIG. 17) from the cholesteryl-aminoxy-lipid 18 and a polyethylene glycol (PEG) derivative (PEG2000-bis-propionaldehyde™) 20 was studied both in solution (chloroform) and in a post-coupling liposome reaction (in water).

In the solution reaction, the aminoxy-lipid 18 and PEG derivative 20 were reacted in a 2:1 ratio and the coupling product 21 was characterised. ¹H NMR analysis shows the appearance in the lower field of two triplets (at 6.88 and 7.60 ppm) corresponding to the two stereoisomers, cis and trans, of the oximes 21. This is also confirmed in ¹³C NMR by the presence of two distinct signals at 151.2 and 151.5 ppm corresponding to the carbon of oxime bond of oximes 21 for the two isomers. This reaction appears to proceed to completion as no remaining aminoxy-compound 18 or PEG₂₀₀₀-bis-propionaldehyde™ 20 was observed.

In the post-coupling reaction, an aminoxy-liposome (DMPC 45%, 18 55%) was reacted in water with PEG₂₀₀₀-bis-propionaldehyde™ 20 also in a 2:1 ratio at pH 4. The product was isolated by chromatography, although the cis and trans stereoisomers were not separable under the chromatographic conditions used. Again, the reaction appeared to proceed to completion. The ¹H NMR, ¹³C NMR and mass spectra showed that the product is the same cholesteryl oxime lipid 21 as obtained from the solution reaction.

A ¹H NMR correlated spectroscopy (¹H-¹H COSY NMR) study was carried out on the product from both the solution reaction (FIG. 18) and the post-coupling liposome reaction (FIGS. 19 and 20). The spectra obtained confirmed the formation of the desired oxime bond.

The reaction can be followed using HPLC (carried out on a Vydac C4 peptide column). Comparision of chromatograms shows that the cholesteryl-aminoxy-lipid 18 gives a peak with a retention time (r.t.) of 25.15 min (see FIG. 14); the peak for PEG₂₀₀₀-bis-propionaldehyde™ 20 has a r.t. of 14.94 min (FIG. 21) and the peak for the product oxime 21 has a r.t. of 31.85 (FIG. 22).

Synthesis of More Complex Molecules: In liposome technologies it is of interest to couple functional moieties directly onto a pre-formed liposomes in aqueous solutions, thereby avoiding complex organic synthesis and purification steps and ensuring that such elements are positioned on the outer layer of the liposomes. Post-coupling methodology generates minimum perturbation of the liposomes.

A series of experiments were performed exemplifing the ability of liposome containing aminoxy-groups to couple onto biologically significant elements. First the aminoxy-group was incorporated onto various lipids, demonstrating the generality of the approach, then, these lipids were inserted into liposomes. Finally, these liposomes were reacted with various biologically significant elements such as sugars, PEGs and glyco-proteins (e.g. blood protein, Transferrin and antibodies) containing either existent or oxidatively generated aldehyde moieties. The effect of the coupling technique could be clearly seen in vivo by studying the modification of the biodistribution profile of the liposomes.

Synthesis of Cholesteryl Aminoxy Lipid 25 (FIG. 23)

Commercially available cholesteryl chloroformate 23 was treated with excess ethylene diamine generating cholesteryl amine 23 Boc-amino-oxyacetic acid was then coupled to amine 23 using HBTU as the coupling reagent affording the Boc-protected cholesteryl aminoxy 24 (81% yield). Deprotection of the Boc-group with HCl in dioxane yielded the product aminoxy lipid 25 (>97% yield by analytical HPLC), which was used without further purification.

Synthesis of DSPE-aminoxy Lipid 29 (FIG. 24)

L-α-disteroyl phosphatidylethanolamine (26, DSPE) (Sigma, UK) was coupled to Boc-amino-oxyacetic acid 27 using HBTU as coupling reagent to afford the Boc-protected DSPE-aminoxy 28 in 56% yield. Removal of the Boc group was achieved with 4M HCl yielding the DSPE-aminoxy lipid 29 (41% yield).

Synthesis of 2-Aminooxy-N-dioctadecylcarbamoylmethyl-acetamide Lipid 35 (FIG. 25)

Dioctadecylamine 30 was coupled to N-Boc glycine 31 using the HBTU reagent affording Boc-protected lipid 32 in 70% yield. Deprotection of the Boc group was achieved using TFA thereby generating amine 33 (92%). Amine 33 was coupled under HBTU conditions to Boc-amino-oxyacetic acid 27 to afford the Boc-protected lipid 34 which was subsequently deprotected with TFA to yield the desired product lipid 35 (85% for 2 steps).

Synthesis of cholesteryl-dPEG₄)₂-aminoxy lipid (CPA) 40 was completed in two stages:
1) the solid phase synthesis of the short protected PEG-aminoxy linker 38 (PABoc, FIG. 26) and,
2) the solution phase coupling of cholesteryl-amine 23 to PABoc 38 (FIG. 27).

PABoc 38 was synthesised on 2-Cholorotrityl chloride polystyrene resin [PS-Chlorotrityl-Cl] (Argonaut, USA) using standard peptide Fmoc solid phase methodology. First, short PEG linker, N-Fmoc-amido-dPEG₄™-acid (Quanta BioDesign, Inc., USA) was loaded onto resin under basic conditions and the Fmoc protecting group subsequently removed with piperidine affording amine 36 (FIG. 26). Next another N-Fmoc-amido-dPEG₄™-acid unit was coupled to 36 using HBTU coupling reagent (Novabiochem, UK) and the Fmoc group subsequently deprotected again. The resultant amine was coupled under HBTU conditions to N-Boc-amino-oxyacetic acid (Novabiochem, UK) affording the resin bound PABoc 37 which was then cleaved from the resin under mild acidic condition to afford crude PABoc 38 which was deemed pure enough (TLC) to continue with the next step without further purification.

PABoc 38 was then coupled to cholesteryl amine 23 using HBTU as the coupling reagent affording the Boc-protected cholesteryl-(dPEG₄)₂-aminoxy 39 (71% yield). Deprotection of the Boc-group with 4M HCl in dioxane yielded the CPA [cholesteryl-(dPEG₄)₂-aminoxy lipid, 40] (>97% yield by analytical HPLC), which was used in biological studies without further purification.

Kinetic of coupling of carbohydrate and polymer on liposomes containing cholesterol-based aminoxy-lipids: Neutral liposomes incorporating an aminoxy-lipid (CholONH₂ 25 or CPA 40) were formulated in water and then incubated with six different reducing sugars or a bis-aldehyde polyethylene glycol (PEG²⁰⁰⁰)(CHO)₂) 20.

Aliquots of the reaction mixture were taken at different times for HPLC analysis to determine the extent of coupling. The reactivity of the aminoxy-lipid was assessed using HPLC, by comparing the surface area of unreacted aminoxy-lipid versus reacted aminoxy-lipid. New peaks in the HPLC were analysed by mass spectrometry to identify the correct coupling product.

FIG. 28 shows the HPLC analysis for the liposome DSPC: CholONH₂ 25 (50:50), where the peak with a retention time (r.t.) of 27.5 min corresponds to CholONH₂ 25, and the broad peak with a r.t. of between 45 and 55 min corresponds to the DSPC. FIGS. 29 and 30 show the HPLC analysis of the reaction of this liposome with lactose and maltoheptaose respectively. The peaks with a r.t. of less than 5 min correspond to the unreacted sugar, and the new peak at 25.75 in FIG. 29, and 24.25 in FIG. 30 correspond to the coupled product.

A graph plotting the course of reactions between liposome 40 and various reduced carbohydrates or PEG²⁰⁰⁰(CHO)₂ are shown in FIG. 31. Similarly reactions with liposome 25 are shown in FIG. 32.

The reactivity of the reducing sugar is dependent of the carbohydrate conformation and the optimal reaction is limited to pH range 3 to 5. The aldehydes of PEG are far more reactive and can be coupled quantitatively at physiological pH. The spacer present on CPA 40 seems to improve its reactivity compared to CholONH₂ 25. The nature of the coupling product of mannose onto a preformed liposome containing CholONH₂ 25 was further confirmed by NMR.

In vivo functionality of a liposomes containing a cholesterol-based aminoxy-lipid modified by lactose or PEG²⁰⁰⁰⁰(CHO)₂: Liposomes incorporating aminoxy-lipid CPA 40 were modified with PEG²⁰⁰⁰(CHO)₂or lactose. The effect on the organ distribution in vivo was analysed. The organ distrubution is represented in FIG. 33. The incubation with lactose resulted in a reduced circulation of the liposome combined with an increased uptake in the liver. This retargeting of the liposome toward the liver is consistent with an increased uptake due to the lactose sensitivity of the asialoglycoreceptor of hepatocytes. The incubation with PEG²⁰⁰⁰(CHO)₂ results in an increased circulation in vivo and a decrease in the liver uptake.

Together these results demonstrate that the in vivo biodistribution of a neutral liposome could be modified using the aminoxy post-coupling technique.

Coupling of carbohydrate and PEG onto liposomes containing a non-cholesterol-based aminoxy-lipids 29 and 35: Two aminoxy-lipids with a non-cholesterol backbone were used to assess if the methodology could be applied to other lipids tails (DSPE-ONH₂29, lipid 35).

The reactivity of the aminoxy-lipid was assessed by comparing the surface area of unreacted aminoxy-lipid versus reacted aminoxy-lipid. New peaks were analysed by mass spectrometry to identify the presence of the correct coupling product. After 24 h, approximately 50% of the lipid 35 and DSPE-ONH₂ 29 was found to be converted in the galactose or PEG coupling product.

Typical HPLC traces are shown in FIG. 34. The trace of a lipid 35: DSPC liposome has a peak with a retention time of 35 mins corresponding to lipid 35 and the broad peak with a retention time of between 41 and 45 mins corresponding to DSPC (see FIG. 34 A). FIG. 34 B is a HPLC trace from the reaction of this liposome and galactose. The peaks for the coupling product (retention time of 36.5 mins) and the unreacted galactose (retention time 4 mins) can be clearly seen. Similarly, FIG. 34 C is a trace from the reaction of the liposome with PEG. The coupling product is observed as a peak at 37 mins, and the unreacted PEG as a peak at 17 min. A mass spectrum of the total liposome fraction for the galactose coupling product is shown in FIG. 35 A, and for the coupling product isolated by HPLC is shown in FIG. 35 B. Similar results were obtained with DSPE-ONH₂29.

These lipids (DSPE-ONH₂ 29, lipid 35) were easily reacted on PEG³⁵⁰⁰(CHO)₂ and a model carbohydrate (galactose). The lipid backbone did not seem to influence the reactivity of the aminoxy group.

Coupling of a protein (transferrin) onto liposomes containing an aminoxy-lipid: A model glycoprotein, transferrin (Tf), was lightly oxidized to generate aldehyde groups on the carbohydrate backbone of the protein. The resulting oxidized-transferrin was then coupled onto a liposome containing an aminoxy-lipid.

HPLC analysis was carried out on liposomes mixed with non-oxidized Tf, Tf oxidized with 10 equivalents of sodium periodate (Tfoxb 10*) and Tf oxidized with 100 equivalents of sodium periodate (Tfox100*). The oxidation of Transferrin does not affect its HPLC profile but does result in a fainter band on the gel. No new peaks could be detected for the non-oxidized Tf-liposome mixture.

A typical result of coupling of Tfox10* onto the liposome is shown in FIGS. 36 and 37. The appearance of a new peak next to the.protein combined to the liposome is clear (FIG. 36). The adsorption of the new peak at 280 nm is in accordance with a protein-based product (FIG. 37).

An enlarged HPLC graph of TfoxlOO* coupled onto the liposome is shown in FIG. 38. Multiple new coupling products are detectable. These products were isolated and was analysed by gel electrophoresis. Their molecular weight is similar to Tf (FIG. 39).

The liposome fraction submitted to free-protein separation (inverted-sucrose gradient) was analysed by HPLC (FIG. 40). The resulting graphs demonstrate the absence of detectable free transferrin in all fractions. No coupling product could be detected by HPLC although such a product would probably be below the detector sensitivity threshold. However this coupling product could be detected in the separated fraction by gel electrophoresis (FIG. 41). Note that no coupling products or free transferrin can be seen in the lane depicting liposome reacted onto non-oxidized transferrin. This result illustrates that only the oxidized transferrin couples onto the liposome and adhere to its surface. Transferrin products are detectable in all non-purified fractions, but only coupling to oxidised transferrin results in transferrin products detectable after purification.

Thus the coupling of the transferrin to the aminoxy-lipid is dependent on the oxidation of the proteins. An increase in the amount of oxidation, resulted in an increase in the amount of coupling products that were detected (FIG. 39). The coupling product of oxidized transferrin and liposome could be separated from free transferrin by a simple inverted sucrose gradient (FIGS. 40 and 41).

Coupling of an antibody onto cationic liposomes containing an aminoxy-lipid (CPA): A rabbit IgG antibody was coupled onto the surface of CDAN/CPA/DOPE (20:30:50, m/m/m) liposomes using a similar methodology as illustrated with transferrin.

As seen in FIGS. 42 and 43 the CPA peak with a retention time of 27 mins decreased by 48% compared to the control where non-oxidized IgG was incubated with liposomes. A new peak with a retention time of 36 mins is observed with a strong absorbance at λ=280nm indicative for proteins, which was isolated and analysed on SDS-page gel and found to be IgG with about 20 copies of CPA covalently bound through the oxidized Fc-carbohydrate units via an oxime bond.

The success of this methodology with both transferrin and Rabbit IgG antibody suggest that it should be generally applicable for all glyco-proteins.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biology, chemistry or related fields are intended to be within the scope of the following claims

1. A process for preparing a modified lipid of the formula 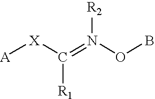
comprising reacting
(i) a compound of the formula; and 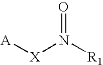
(ii) a compound of the formula 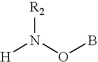
wherein component (ii) is formulated as a liposome or as a component of a liposome;
wherein B is a lipid;
wherein A is a moiety of interest (MOI) and is a hydrocarbyl group;
wherein X is an optional linker group;
wherein R₁ is H or a hydrocarbyl group; and
wherein R₂ is a lone pair, H or a hydrocarbyl group.

2. The process according to claim 1 wherein components (i) and (ii) are in admixture with or associated with a nucleotide sequence, or a pharmaceutically active agent.

3. The process according to claim 1 wherein the reaction is performed in an aqueous medium.

4. The process according to claim 1 wherein A is selected from a carbohydrate moiety, a polymer, a peptide, a glycoprotein, a small biomolecule and a bioconjugate linker.

5. The process according to claim 4 wherein A is a small biomolecule selected from folic acid and a folic acid derivative.

6. The process according to claim 4 wherein A is a carbohydrate moiety selected from mannose, glucose (D-glucose), galactose, glucuronic acid, lactose, maltose, maltotriose, maltotetraose, maltoheptaose and mixtures thereof.

7. The process according to claim 4 wherein A is a polyether polymer.

8. The process according to claim 7 wherein A is a polyethylene glycol.

9. The process according to claim 4 wherein A is a bioconjugate linker selected from an aldehyde, an amine, a thiocyanate, an isocyanate and a maleimide group.

10. The process according to claim 1 wherein A is transferrin.

11. The process according to claim 1 wherein A is an antibody.

12. The process according to claim 1 wherein A comprises an RGD peptide.

13. The process according to claim 1 wherein B comprises a lipid of the formula:
-W-Y-Z;
wherein W comprises a group selected from a polyamine group, a polyether group and mixtures thereof;
wherein Y is a linkage group; and
wherein Z is selected from a steroid, an acyl glcerol, a phosphoglceride, a ceramide and an acetamide derivative.

14. The process according to claim 13 wherein W comprises a polyamine group.

15. The process according to claim 14 wherein the polyamine group contains at least two amines of the polyamine group that are separated (spaced from each other) from each other by an ethylene (—CH₂CH₂—) group.

16. The process according to claim 14 wherein the polyamine group is selected from spermidine, spermine, caldopentamine, norspermidine and norspermine.

17. The process according to claim 13 wherein W comprises a polyether group.

18. The process according to claim 17 wherein the polyether group is a polyethylene glycol (PEG) polymer.

19. The process according to claim 13 wherein Y is a linkage group selected from an ester, amide, carbamate and ether group.

20. The process according to claim 13 wherein Z is a steroid.

21. The process according to claim 20 wherein the steroid is cholesterol.

22. The process according to claim 13 wherein Z comprises an acetamide derivative.

23. The process according to claim 22 wherein the acetamide derivative is a dialkyl substituted acetamide derivative of the formula —C(O)—NR₁₀R₁₁,
wherein R₁₀ and R₁₁ are independently selected from H and a long chain hydrocarbyl group.

24. The process of claim 1 wherein R₁ is H.

25. The process of claim 1 wherein R₂ is H.

26. The process of claim 1 wherein X is a hydrocarbyl group.

27. The process of claim 1 wherein X is a polyether group.

28. A process for preparing a compound of the formula 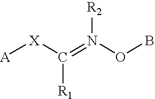
comprising reacting
(i) a compound of the formula; and 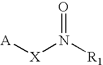
(ii) a compound of the formula 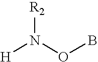
in admixture with or associated with a nucleotide sequence, or a pharmaceutically active agent;
wherein B is a lipid;
wherein A is a moiety of interest (MOI) and is a hydrocarbyl group;
wherein X is an optional linker group;
wherein R₁ is H or a hydrocarbyl group; and
wherein R₂ is a lone pair, H or a hydrocarbyl group.

29. A composition comprising
(i) a compound of the formula 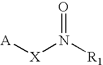
(ii) a compound of the formula 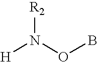
wherein component (ii) is formulated as a liposome or as a component of a liposome;
wherein B is a lipid;
wherein A is a moiety of interest (MOI) and is a hydrocarbyl group;
wherein X is an optional linker group;
wherein R₁ is H or a hydrocarbyl group; and
wherein R₂ is a lone pair, H or a hydrocarbyl group.

30. The composition according to claim 28 further comprising (iii) a nucleotide sequence, or a pharmaceutically active agent.

31. A composition comprising
(i) a compound of the formula 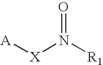
(ii) a compound of the formula 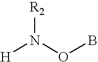
and
(iii) a nucleotide sequence, or a pharmaceutically active agent;
wherein B is a lipid;
wherein A is a moiety of interest (MOI) and is a hydrocarbyl group;
wherein X is an optional linker group;
wherein R₁ is H or a hydrocarbyl group; and
wherein R₂ is a lone pair, H or a hydrocarbyl group.
